(19) United States
(12) Patent Application Publication
(10) Pub. No.: US 2020/0040405 A1
(43) Pub. Date: Feb. 6, 2020

(54) COLORECTAL CANCER SCREENING METHOD AND DEVICE

(71) Applicant: GENEOSCOPY, LLC, St. Louis, MO (US)

(72) Inventors: Erica BARNELL, St. Louis, MO (US); Marianne LIGON, St. Louis, MO (US); Yiming KANG, Clayton, MO (US)

(73) Assignee: GENEOSCOPY, LLC, St. Louis, MO (US)

(21) Appl. No.: 16/593,034

(22) Filed: Oct. 4, 2019

Related U.S. Application Data
(63) Continuation of application No. 15/570,507, filed on Oct. 30, 2017.
(60) Provisional application No. 62/154,506, filed on Apr. 29, 2015.

(51) Int. Cl. C12Q 1/6886

(52) U.S. Cl. CPC C12Q 1/6886; C12Q 2600/118; C12Q 2600/158; C12Q 2600/112; C12Q 2600/106

(57) ABSTRACT

Provided herein are compositions and methods for diagnosis and treatment of colorectal cancer. Methods and kits for detection of colorectal cancer biomarker genes in a stool sample are provided.

### CROSS REFERENCE TO RELATED APPLICATIONS

This application is a continuation of U.S. application Ser. No. 15/570,507, which was filed on Oct. 30, 2017, which is a U.S. national phase application filed under 35 U.S.C. § 371 of International Application No. PCT/US2016/029777, which was filed Apr. 28, 2016, and which claims the benefit of the filing date of U.S. Provisional Patent Application No. 62/154,506, which was filed Apr. 29, 2015. The entire content of these applications is hereby incorporated by reference herein.

### FIELD OF INVENTION

The present invention relates to the diagnosis and treatment of colorectal cancer.

### BACKGROUND

Colorectal cancer (CRC) is the third most common cancer among both men and women. In the United States, colorectal cancer is the second leading cause of cancer-related death, killing over 51,000 men and women annually. The National Cancer Institute estimates that more than 130,000 new cases of colorectal cancer were diagnosed in the US in 2015. The Center for Disease Control estimates that in 2012, the last year for which statistics are available, there were approximately 1.4 million new cases of colorectal cancer and approximately 694,000 deaths worldwide. In the US, both incidence and death rates have been decreasing. These decreases over the past decade have generally been attributed to the detection and removal of precancerous polyps as a result of increased colorectal cancer screening. However, existing screening methods remain problematic. Colonoscopy is considered the “gold standard” for detecting colorectal cancer due to its diagnostic accuracy. However, colonoscopies are invasive, they require an extensive time commitment by the patient, they include pre-procedural steps that discourage patient compliance in obtaining timely test results, and they are associated with relatively high costs. Other invasive tests such as CT colonography and barium enemas have similar drawbacks and are not as diagnostically accurate as colonoscopy. Noninvasive methods, for example fecal DNA tests, fecal immunochemical tests, and fecal occult blood tests generally lack the accuracy of more invasive methods. There is a continuing need for methods of screening and diagnosis of colorectal cancer.

### SUMMARY

Provided herein are methods and compositions for detection of colorectal cancer. The method of detection of colorectal cancer in a subject can include a) measuring the level of expression of two or more colorectal cancer biomarker genes selected from any of the colorectal cancer biomarker genes listed in Table 1 (Panel A) in a biological sample from the subject; b) comparing the measured expression level of the two or more colorectal cancer biomarker genes in the sample with the measured expression level of the two or more colorectal cancer biomarker genes in a control sample, wherein a difference in the measured expression level of the two more genes in the biological sample relative to the measured expression level of the two or more genes in the control sample indicates that the subject has colorectal cancer. The two or more colorectal cancer biomarker genes can be selected from the colorectal cancer biomarker genes listed in Panel B, Panel C, Panel D, or Panel E. The two or more colorectal cancer biomarker genes are selected from the group consisting of AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1.

The method can include providing a biological sample from the subject. The biological sample can be a stool sample. The expression level can include expression of an RNA selected from the group consisting of total RNA, mRNA, tRNA, rRNA, ncRNA, smRNA, and snoRNA. In one aspect, the measuring step comprises microarray analysis, reverse transcription polymerase chain reaction (RT-PCR), or nucleic acid sequencing. In one aspect, the control sample can include a reference value.

In some embodiments, the colorectal cancer is selected from the group consisting of Stage 1(T1), Stage 2 (T2), Stage 3 (T-3), and Stage 4 (T4). The colorectal cancer can be a tubular adenocarcinoma, a villous adenocarcinoma, a gastrointestinal stromal tumor, a primary colorectal lymphoma, a leiomysarcoma, melanoma, a squamous cell carcinoma, or a mucinous carcinoma.

Also provided are methods of determining whether a subject is at risk for colorectal cancer. The method of determining whether a subject is at risk for colorectal cancer can include: a) measuring the level of expression of two or more colorectal cancer biomarker genes selected from any of the colorectal cancer biomarker genes listed in Table 1 (Panel A) in a biological sample from the subject; b) comparing the measured expression level of the two or more colorectal cancer biomarker genes in the sample with the measured expression level of the two or more colorectal cancer biomarker genes in a control sample, wherein a difference in the measured expression level of the two or more genes in the biological sample relative to the measured expression level of the two or more genes in the control sample indicates that the subject is at risk for colorectal cancer. The two or more colorectal cancer biomarker genes can be selected from the colorectal cancer biomarker genes listed in Panel B, Panel C, Panel D, or Panel E. The two or more colorectal cancer biomarker genes are selected from the group consisting of AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1.

The method can include providing a biological sample from the subject. The biological sample can be a stool sample. The expression level can include expression of an RNA selected from the group consisting of total RNA, mRNA, tRNA, rRNA, ncRNA, smRNA, and snoRNA. In one aspect, the measuring step comprises microarray analysis, reverse transcription polymerase chain reaction (RT-PCR), or nucleic acid sequencing. In one aspect, the control sample can include a reference value.

In some embodiments, the colorectal cancer is selected from the group consisting of Stage 1(T1), Stage 2 (T2), Stage 3 (T-3), and Stage 4 (T4). The colorectal cancer can be a tubular adenocarcinoma, a villous adenocarcinoma, a gastrointestinal stromal tumor, a primary colorectal lymphoma, a leiomysarcoma, melanoma, a squamous cell carcinoma, or a mucinous carcinoma.

Also provided is a method of selecting a clinical plan for a subject having or at risk for colorectal cancer. The method of selecting a clinical plan for a subject having or at risk for colorectal cancer can include: a) measuring the level of expression of two or more colorectal cancer biomarker genes selected from any of the colorectal cancer biomarker genes listed in Table 1 (Panel A) in a biological sample from the subject; b) comparing the measured expression level of the two or more colorectal cancer biomarker genes in the sample with the measured expression level of the two or more colorectal cancer biomarker genes in a control sample, wherein a difference in the measured expression level of the two or more genes relative to the measured expression level of the two or more genes in the control sample indicates that the subject has or is at risk for colorectal cancer; and c) selecting a clinical plan based on step b. The two or more colorectal cancer biomarker genes can be selected from the colorectal cancer biomarker genes listed in Panel B, Panel C, Panel D, or Panel E. The two or more colorectal cancer biomarker genes are selected from the group consisting of AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1.

The method can include providing a biological sample from the subject. The biological sample can be a stool sample. The expression level can include expression of an RNA selected from the group consisting of total RNA, mRNA, tRNA, rRNA, ncRNA, smRNA, and snoRNA. In one aspect, the measuring step comprises microarray analysis, reverse transcription polymerase chain reaction (RT-PCR), or nucleic acid sequencing. In one aspect, the control sample can include a reference value.

In some embodiments, the colorectal cancer is selected from the group consisting of Stage 1(T1), Stage 2 (T2), Stage 3 (T-3), and Stage 4 (T4). The colorectal cancer can be a tubular adenocarcinoma, a villous adenocarcinoma, a gastrointestinal stromal tumor, a primary colorectal lymphoma, a leiomysarcoma, melanoma, a squamous cell carcinoma, or a mucinous carcinoma.

In one aspect, the clinical plan comprises a diagnostic procedure or a treatment. The diagnostic procedure can include a fecal occult blood test, a fecal immunochemical test, or a colonoscopy. The treatment can include surgery, chemotherapy, radiation therapy, targeted therapy, or immunotherapy. The chemotherapy can include administration of 5-fluorouracil, leucovorin, capecitabine, oxaliplatin, irinotecan or a combination thereof. The targeted therapy can include administration of bevacizumab (anti-VEGF), ramuciramab (anti-VEGFR2), aflibercept, regorafenib, cetuximab (anti-EGFR), panitumumab, tripfluridine-tipiracil or a combination thereof.

Also provided is a panel of colorectal cancer biomarker genes comprising AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1

Also provided are sets of detectably labeled probes to a panel of biomarkers. In one aspect, the detectably labeled probes can include probes to a panel of biomarkers comprising AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1.

Also provided are kits. In one aspect, a kit can include: a) a set of detectably labeled probes to a panel of colorectal cancer biomarkers comprising AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1 and b) two or more items selected from the group consisting of control nucleic acids corresponding to a panel of biomarkers comprising AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1, packaging material, a package insert comprising instructions for use, a sterile fluid, a syringe, and a sterile container.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 is a heat map analysis of the 564 colorectal cancer biomarker genes listed in Table 1 (Panel A).

FIG. 2: is a heat map analysis of the 277 colorectal cancer biomarker genes listed in Panel B.

FIG. 3 is a heat map analysis of the 95 colorectal cancer biomarker genes listed in Panel C.

FIG. 4 is a heat map analysis of the 39 colorectal cancer biomarker genes listed in Panel D.

FIG. 5 is a heat map analysis of the 22 colorectal cancer biomarker genes listed in Panel E.

FIG. 6: shows the results of a principal component analysis of the colorectal cancer biomarker genes listed in Table 1.

### DETAILED DESCRIPTION

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. When only a single machine is illustrated, the term “machine” shall also be taken to include any collection of machines that individually or jointly execute a set (or multiple sets) of instructions to perform any one or more of the methodologies discussed herein. In the claims, means-plus-function clauses, if used, are intended to cover the structures described, suggested, or rendered obvious by the written description or drawings for performing the recited function, including not only structural equivalents but also equivalent structures.

The present invention is based in part on our discovery that we could separate human cells from bacterial cells in a human stool sample in order to obtain human RNA that was enriched for human nucleic acids thereby allowing detection of human colorectal cancer biomarker genes in a stool sample. Accordingly, provided herein are methods and compositions for determining whether a subject is suffering from or is at risk for colorectal cancer. The methods and compositions are also useful for selecting a clinical plan for a subject suffering from colorectal cancer. The clinical plan can include administration of further diagnostic procedures. In some embodiments, the clinical plan can include a method of treatment. The methods include detection of colorectal cancer in a subject. The methods can include methods of isolation of human RNA from a stool sample obtained from a subject. The methods can include determining the level of expression of two or more colorectal cancer biomarker genes in the human RNA isolated from a stool sample obtained from a patient and determining whether the levels of the two or more colorectal cancer biomarker genes are different relative to the levels of the same two or more colorectal cancer biomarker genes in a control sample. The colorectal cancer biomarker genes can include two or more of any of the colorectal cancer biomarker genes shown in Table 1. All of the colorectal cancer biomarker genes listed in Table 1 form a panel (“Panel A”). The colorectal cancer biomarker genes in Table 1 can also include subsets of colorectal cancer biomarker genes, for example, Panels, B, C, D, and E. The compositions can include gene arrays and probe sets configured for the specific detection of the panels of markers disclosed herein. The compositions can also include kits comprising gene arrays and probe sets configured for the specific detection of the panels of markers disclosed herein.

TABLE 1
Colorectal cancer biomarker genes

| | | NCBI or Ensembl | |
|---|---|---|---|
| Gene Symbol | Gene Description | Accession Number | Panel |
| — | — | AK024621 | A, B, C, D and E |
| SNORD51 | small nucleolar RNA, | NR_002589 | A, B, C, D and E |
| | C/D box 51 | | |
| — | — | TCONS_l2_00011049- | A, B, C, D and E |
| | | XLOC_l2_005952 | |
| PRTG | protogenin | AK022857 | A, B, C, D and E |
| MIR933 | microRNA 933 | NR_030630 | A, B, C, D and E |
| ID1 | inhibitor of DNA | NM_002165 | A, B, C, D and E |
| | binding 1, dominant | | |
| | negative helix-loop- | | |
| | helix protein | | |
| — | — | ENST00000459148 | A, B, C, D and E |
| PCDHB18 | protocadherin beta 18 | NR_001281 | A, B, C, D and E |
| | pseudogene | | |
| RP11-23D5.1 | putative novel | OTTHUMT00000051727 | A, B, C, D and E |
| | transcript | | |
| RNU6-716P | RNA, U6 small | ENST00000365621 | A, B, C, D and E |
| | nuclear 716, | | |
| | pseudogene | | |
| — | — | BC039358 | A, B, C, D and E |
| OR5V1 | olfactory receptor, | NM_030876 | A, B, C, D and E |
| | family 5, subfamily V, | | |
| | member 1 | | |
| IGLV7-43 | immunoglobulin | ENST00000390298 | A, B, C, D and E |
| | lambda variable 7-43 | | |
| — | — | TCONS_00014878- | A, B, C, D and E |
| | | XLOC_006946 | |
| — | — | TCONS_00028807- | A, B, C, D and E |
| | | XLOC_013883 | |
| — | — | linc_luo_1487 | A, B, C, D and E |
| — | — | TCONS_l2_00017903- | A, B, C, D and E |
| | | XLOC_l2_009470 | |
| — | — | TCONS_00009728- | A, B, C, D and E |
| | | XLOC_004927 | |
| — | — | ENST00000408390 | A, B, C, D and E |
| — | — | ENST00000384552 | A, B, C, D and E |
| — | — | uc021uck.1 | A, B, C, D and E |
| — | — | TCONS_00017621- | A, B, C, and D |
| | | XLOC_008311 | |
| — | — | ENST00000364506 | A, B, C, and D |
| KISS1R | KISS1 receptor | NM_032551 | A, B, C, and D |
| — | — | ENST00000554665 | A, B, C, and D |
| — | — | AF086063 | A, B, C, and D |
| — | — | ENST00000528885 | A, B, C, and D |
| MIR4474 | microRNA 4474 | NR_039685 | A, B, C, and D |
| — | — | ENST00000557910 | A, B, C, and D |
| DNM1L | dynamin 1-like | AK090788 | A, B, C, and D |
| LOC401242 | uncharacterized | NR_033379 | A, B, C, and D |
| | LOC401242 | | |
| — | — | ENST00000384633 | A, B, C, and D |
| RP11-15B24.5 | novel transcript | OTTHUMT00000052823 | A, B, C, and D |
| PANK2 | pantothenate kinase 2 | BC008667 | A, B, C, and D |
| GFRAL | GDNF family receptor | NM_207410 | A, B, C, and D |
| | alpha like | | |
| OR2L2 | olfactory receptor, | X64978 | A, B, C, and D |
| | family 2, subfamily L, | | |
| | member 2 | | |
| — | — | TCONS_00028080- | A, B, C, and D |
| | | XLOC_013828 | |
| RNU6-572P | RNA, U6 small | ENST00000516724 | A, B, C, and D |
| | nuclear 572, | | |
| | pseudogene | | |
| RNU6-316P | RNA, U6 small | ENST00000391027 | A, B, and C |
| | nuclear 316, | | |
| | pseudogene | | |
| — | — | ENST00000411365 | A, B, and C |
| RP11-219F10.1 | putative novel | OTTHUMT00000049107 | A, B, and C |
| | transcript | | |
| — | — | TCONS_l2_00030381- | A, B, and C |
| | | XLOC_l2_015636 | |
| — | — | DQ584116 | A, B, and C |
| — | — | ENST00000384011 | A, B, and C |
| — | — | DQ593444 | A, B, and C |
| AFF2-IT1 | AFF2 intronic | ENST00000435346 | A, B, and C |
| | transcript 1 (non- | | |
| | protein coding) | | |
| OR5V1 | olfactory receptor, | OTTHUMT00000309673 | A, B, and C |
| | family 5, subfamily V, | | |
| | member 1 | | |
| MIR4796 | microRNA 4796 | NR_039959 | A, B, and C |
| OR5V1 | olfactory receptor, | NM_030876 | A, B, and C |
| | family 5, subfamily V, | | |
| | member 1 | | |
| — | — | TCONS_l2_00014322- | A, B, and C |
| | | XLOC_l2_007828 | |
| — | — | DQ587050 | A, B, and C |
| MIR516B1 | microRNA 516b-1 | NR_030212 | A, B, and C |
| AC114803.3 | novel transcript | OTTHUMT00000335541 | A, B, and C |
| — | — | ENST00000459507 | A, B, and C |
| — | — | uc022ayv.1 | A, B, and C |
| TNRC6C | trinucleotide repeat | BC039479 | A, B, and C |
| | containing 6C | | |
| ZNF256 | zinc finger protein 256 | NM_005773 | A, B, and C |
| — | — | DQ589981 | A, B, and C |
| — | — | uc022avm.1 | A, B, and C |
| RNU6-31P | RNA, U6 small | ENST00000384388 | A, B, and C |
| | nuclear 31, | | |
| | pseudogene | | |
| AL022344.4 | novel transcript | OTTHUMT00000047687 | A, B, and C |
| — | — | ENST00000516036 | A, B, and C |
| DUX2 | double homeobox 2 | NM_012147 | A, B, and C |
| — | — | ENST00000555316 | A, B, and C |
| RP11-451B8.1 | novel transcript | OTTHUMT00000352848 | A, B, and C |
| — | — | ENST00000391095 | A, B, and C |
| DXO | decapping | AF059253 | A, B, and C |
| | exoribonuclease | | |
| LOC90784 | uncharacterized | AK001612 | A, B, and C |
| | LOC90784 | | |
| RP1-92C4.2 | putative novel | OTTHUMT00000041312 | A, B, and C |
| | transcript | | |
| LOC101927138 | uncharacterized | ENST00000412519 | A, B, and C |
| | LOC101927138 | | |
| MIR644A | microRNA 644a | NR_030374 | A, B, and C |
| MIR661 | microRNA 661 | NR_030383 | A, B, and C |
| — | — | ENST00000516983 | A, B, and C |
| AC064865.1 | novel transcript | OTTHUMT00000332167 | A, B, and C |
| SRR | serine racemase | AY743705 | A, B, and C |
| — | — | Z97017 | A, B, and C |
| SNORD127 | small nucleolar RNA, | NR_003691 | A, B, and C |
| | C/D box 127 | | |
| LOC401242 | uncharacterized | NR_033379 | A, B, and C |
| | LOC401242 | | |
| MIR589 | microRNA 589 | NR_030318 | A, B, and C |
| — | — | TCONS_00011937- | A, B, and C |
| | | XLOC_005448 | |
| — | — | TCONS_00029494- | A, B, and C |
| | | XLOC_014412 | |
| APLNR | apelin receptor | NR_027991 | A, B, and C |
| RP4-584D14.6 | putative novel | OTTHUMT00000350703 | A, B, and C |
| | transcript | | |
| — | — | BC038672 | A, B, and C |
| GFER | growth factor, | NM_005262 | A, B, and C |
| | augmenter of liver | | |
| | regeneration | | |
| — | — | TCONS_00018151- | A, B, and C |
| | | XLOC_008430 | |
| RNA5SP319 | RNA, 5S ribosomal | ENST00000362768 | A, B, and C |
| | pseudogene 319 | | |
| — | — | ENST00000408662 | A, B, and C |
| — | — | DQ597648 | A, B, and C |
| — | — | DQ576504 | A, B, and C |
| TGFB1 | transforming growth | NM_000660 | A, B, and C |
| | factor, beta 1 | | |
| — | — | BC024025 | A, B, and C |
| RNU6-281P | RNA, U6 small | ENST00000384212 | A, B, and C |
| | nuclear 281, | | |
| | pseudogene | | |
| RN7SKP252 | RNA, 7SK small | ENST00000411210 | A, B, and C |
| | nuclear pseudogene | | |
| | 252 | | |
| C8orf17 | chromosome 8 open | AF220264 | A and B |
| | reading frame 17 | | |
| CTD- | novel transcipt | OTTHUMT00000369511 | A and B |
| 2116N20.1 | | | |
| LOC101927138 | uncharacterized | BC033543 | |
| | LOC101927138 | | |
| — | — | AL110200 | A and B |
| RP11- | novel transcript | OTTHUMT00000047851 | A and B |
| 144G6.10 | | | |
| — | — | linc_luo_1768 | A and B |
| — | — | BC036682 | A and B |
| RP11-168P8.3 | putative novel | OTTHUMT00000047733 | A and B |
| | transcript | | |
| RP11-600L4.1 | putative novel | OTTHUMT00000360544 | A and B |
| | transcript | | |
| RNU7-110P | RNA, U7 small | ENST00000516891 | A and B |
| | nuclear 110 | | |
| | pseudogene | | |
| SNORD115-4 | small nucleolar RNA, | NR_003296 | A and B |
| | C/D box 115-4 | | |
| — | — | AY863198 | A and B |
| — | — | ENST00000560324 | A and B |
| MIR380 | microRNA 380 | NR_029872 | A and B |
| — | — | ENST00000364957 | A and B |
| MIR4508 | microRNA 4508 | NR_039731 | A and B |
| MIR4476 | microRNA 4476 | NR_039687 | A and B |
| CTD-2023M8.1 | novel transcript | OTTHUMT00000366267 | A and B |
| RBSG2 | retinoblastoma-specific | AB593131 | A and B |
| | gene 2 | | |
| — | — | ENST00000362696 | A and B |
| — | — | ENST00000408425 | A and B |
| RNU6-1310P | RNA, U6 small | ENST00000384153 | A and B |
| | nuclear 1310, | | |
| | pseudogene | | |
| RP11-13P5.1 | novel transcript | OTTHUMT00000042895 | A and B |
| — | — | TCONS_00024446- | A and B |
| | | XLOC_011769 | |
| PTPRS | protein tyrosine | S78080 | A and B |
| | phosphatase, receptor | | |
| | type, S | | |
| — | — | BC036204 | A and B |
| LOC401242 | uncharacterized | NR_033379 | A and B |
| | LOC401242 | | |
| — | — | ENST00000384103 | A and B |
| ZBTB12 | zinc finger and BTB | NM_181842 | A and B |
| | domain containing 12 | | |
| CTD- | novel transcript | OTTHUMT00000366755 | A and B |
| 2333M24.1 | | | |
| — | — | TCONS_00028865- | A and B |
| | | XLOC_013999 | |
| — | — | TCONS_l2_00011482- | A and B |
| | | XLOC_l2_006206 | |
| — | — | ENST00000547795 | A and B |
| RP11-561I11.2 | — | OTTHUMT00000096192 | A and B |
| TRPC3 | transient receptor | X89068 | A and B |
| | potential cation | | |
| | channel, subfamily C, | | |
| | member 3 | | |
| C8orf17 | chromosome 8 open | ENST00000507535 | A and B |
| | reading frame 17 | | |
| KRTAP10-7 | keratin associated | NM_198689 | A and B |
| | protein 10-7 | | |
| — | — | TCONS_l2_00021363- | A and B |
| | | XLOC_l2_011322 | |
| — | — | ENST00000384305 | A and B |
| C17orf100 | chromosome 17 open | NM_001105520 | A and B |
| | reading frame 100 | | |
| RNU2-42P | RNA, U2 small | ENST00000410697 | A and B |
| | nuclear 42, | | |
| | pseudogene | | |
| — | — | AF399612 | A and B |
| ROR1 | receptor tyrosine | AK000776 | A and B |
| | kinase-like orphan | | |
| | receptor 1 | | |
| — | — | ENST00000408143 | A and B |
| LINC00112 | long intergenic non- | NR_024028 | A and B |
| | protein coding RNA | | |
| | 112 | | |
| OR5V1 | olfactory receptor, | NM_030876 | A and B |
| | family 5, subfamily V, | | |
| | member 1 | | |
| — | — | DQ588149 | A and B |
| RP11-15G16.1 | novel transcript | OTTHUMT00000377136 | A and B |
| RP5-881L22.5 | novel transcript, | OTTHUMT00000079346 | A and B |
| | antisense to R3HDML | | |
| — | — | uc003kgf.1 | A and B |
| — | — | TCONS_l2_00007465- | A and B |
| | | XLOC_l2_003848 | |
| D21S2088E | D21S2088E | NR_040254 | A and B |
| SNRK-AS1 | SNRK antisense RNA 1 | ENST00000422681 | A and B |
| — | — | CR606964 | A and B |
| HBA2 | hemoglobin, alpha 2 | DQ655927 | A and B |
| LOC101929350 | uncharacterized | ENST00000422917 | A and B |
| | LOC101929350 | | |
| RP11-233E12.1 | novel transcript | OTTHUMT00000001239 | A and B |
| — | — | uc021wsq.1 | A and B |
| RP11- | novel transcript | OTTHUMT00000041583 | A and B |
| 436D23.1 | | | |
| CD8A | CD8a molecule | NR_027353 | A and B |
| — | — | DQ582489 | A and B |
| IGKC | immunoglobulin kappa | X72451 | A and B |
| | constant | | |
| — | — | ENST00000555465 | A and B |
| — | — | ENST00000517282 | A and B |
| — | — | DQ575530 | A and B |
| — | — | DQ591628 | A and B |
| OR1J1 | olfactory receptor, | NM_001004451 | A and B |
| | family 1, subfamily J, | | |
| | member 1 | | |
| — | — | DQ591298 | A and B |
| — | — | ENST00000458902 | A and B |
| — | — | TCONS_l2_00030165- | A and B |
| | | XLOC_l2_015472 | |
| — | — | TCONS_00024376- | A and B |
| | | XLOC_011699 | |
| — | — | ENST00000554623 | A and B |
| OR1D4 | olfactory receptor, | NR_033795 | A and B |
| | family 1, subfamily D, | | |
| | member 4 | | |
| | (gene/pseudogene) | | |
| H2BFWT | H2B histone family, | NM_001002916 | A and B |
| | member W, testis- | | |
| | specific | | |
| — | — | ENST00000557687 | A and B |
| — | — | AK130206 | A and B |
| — | — | linc_luo_1651 | A and B |
| — | — | uc003zmg.2 | A and B |
| RNU6-1176P | RNA, U6 small | ENST00000390955 | A and B |
| | nuclear 1176, | | |
| | pseudogene | | |
| — | — | TCONS_l2_00003921- | A and B |
| | | XLOC_l2_001518 | |
| — | — | DQ589683 | A and B |
| HNRNPM | heterogeneous nuclear | BC038753 | A and B |
| | ribonucleoprotein M | | |
| BTBD18 | BTB (POZ) domain | NM_001145101 | A and B |
| | containing 18 | | |
| LINC00086 | long intergenic non- | BC030620 | A and B |
| | protein coding RNA | | |
| | 86 | | |
| KRTAP1-5 | keratin associated | NM_031957 | A and B |
| | protein 1-5 | | |
| — | — | trnA | A and B |
| — | — | ENST00000555016 | A and B |
| — | — | uc021tdf.1 | A and B |
| — | — | TCONS_00006525- | A and B |
| | | XLOC_003150 | |
| — | — | ENST00000546982 | A and B |
| — | — | OTTHUMT00000365271 | A and B |
| LOC100130238 | uncharacterized | uc010tbp.1 | A and B |
| | LOC100130238 | | |
| RNU6-175P | RNA, U6 small | ENST00000516896 | A and B |
| | nuclear 175, | | |
| | pseudogene | | |
| MIR635 | microRNA 635 | NR_030365 | A and B |
| — | — | TCONS_00001278- | A and B |
| | | XLOC_000566 | |
| ZNF71 | zinc finger protein 71 | NM_021216 | A and B |
| — | — | DQ600483 | A and B |
| RNU6-528P | RNA, U6 small | ENST00000516926 | A and B |
| | nuclear 528, | | |
| | pseudogene | | |
| — | — | linc_luo_876 | A and B |
| — | — | BC134347 | A and B |
| RNA5SP84 | RNA, 5S ribosomal | ENST00000364740 | A and B |
| | pseudogene 84 | | |
| LY6G6D | lymphocyte antigen 6 | AJ315537 | A and B |
| | complex, locus G6D | | |
| RP11-440G9.1 | novel transcript | OTTHUMT00000042494 | A and B |
| RABGAP1L- | RABGAP1L intronic | ENST00000414890 | A and B |
| IT1 | transcript 1 (non- | | |
| | protein coding) | | |
| LOC101926908 | uncharacterized | ENST00000519427 | A and B |
| | LOC101926908 | | |
| — | — | ENST00000557745 | A and B |
| — | — | TCONS_l2_00003545- | A and B |
| | | XLOC_l2_001961 | |
| — | — | AK123915 | A and B |
| — | — | AF344194 | A and B |
| — | — | TCONS_00015793- | A and B |
| | | XLOC_007646 | |
| CTD- | novel transcript, | OTTHUMT00000365493 | A and B |
| 2194D22.3 | antisense to IRX4 | | |
| — | — | ENST00000532913 | A and B |
| — | — | DQ597441 | A and B |
| — | — | TCONS_00018037- | A and B |
| | | XLOC_008938 | |
| — | — | uc002dam.1 | A and B |
| CSH1 | chorionic | NM_001317 | A and B |
| | somatomammotropin | | |
| | hormone 1 (placental | | |
| | lactogen) | | |
| CCSAP | centriole, cilia and | BC039241 | A and B |
| | spindle-associated | | |
| | protein | | |
| — | — | ENST00000557152 | A and B |
| — | — | TCONS_00021771- | A and B |
| | | XLOC_010367 | |
| — | — | TCONS_00009616- | A and B |
| | | XLOC_004750 | |
| — | — | TCONS_00000453- | A and B |
| | | XLOC_000676 | |
| ERICH5 | glutamate-rich 5 | NM_001170806 | A and B |
| — | — | DQ576853 | A and B |
| UNC5C | unc-5 homolog C (C. elegans) | BX538341 | A and B |
| — | — | ENST00000555514 | A and B |
| OR6C75 | olfactory receptor, | NM_001005497 | A and B |
| | family 6, subfamily C, | | |
| | member 75 | | |
| — | — | TCONS_00003265- | A and B |
| | | XLOC_002069 | |
| AC084809.2 | novel transcript | OTTHUMT00000256183 | A and B |
| — | — | linc_luo_1664 | A and B |
| — | — | ENST00000515991 | A and B |
| RNU6-1058P | RNA, U6 small | ENST00000516392 | A and B |
| | nuclear 1058, | | |
| | pseudogene | | |
| — | — | TCONS_00015650- | A and B |
| | | XLOC_007286 | |
| CROCCP2 | ciliary rootlet coiled- | BC127868 | A and B |
| | coil, rootletin | | |
| | pseudogene 2 | | |
| — | — | TCONS_00015728- | A and B |
| | | XLOC_007495 | |
| — | — | ENST00000454160 | A and B |
| — | — | AF085988 | A and B |
| LOC101927000 | uncharacterized | ENST00000453149 | A and B |
| | LOC101927000 | | |
| — | — | uc021ymw.1 | A and B |
| — | — | ENST00000410619 | A and B |
| RAB1B | RAB1B, member RAS | ENST00000501708 | A and B |
| | oncogene family | | |
| TMEM42 | transmembrane protein | NM_144638 | A and B |
| | 42 | | |
| RNU6-916P | RNA, U6 small | ENST00000516088 | A and B |
| | nuclear 916, | | |
| | pseudogene | | |
| RNU6-615P | RNA, U6 small | ENST00000516065 | A and B |
| | nuclear 615, | | |
| | pseudogene | | |
| DEFB113 | defensin, beta 113 | NM_001037729 | A and B |
| — | — | DQ585964 | A and B |
| — | — | DQ585964 | A and B |
| — | — | ENST00000560068 | A and B |
| — | — | TCONS_00016129- | A and B |
| | | XLOC_007516 | |
| RNU11 | RNA, U11 small | NR_004407 | A and B |
| | nuclear | | |
| — | — | ENST00000499173 | A and B |
| RNU6-523P | RNA, U6 small | ENST00000516304 | A and B |
| | nuclear 523, | | |
| | pseudogene | | |
| RP11- | novel transcript | OTTHUMT00000362023 | A and B |
| 161D15.2 | | | |
| — | — | X07060 | A and B |
| — | — | TCONS_00007656- | A and B |
| | | XLOC_003732 | |
| — | — | TCONS_l2_00004945- | A and B |
| | | XLOC_l2_002603 | |
| RNU6-847P | RNA, U6 small | ENST00000411115 | A and B |
| | nuclear 847, | | |
| | pseudogene | | |
| — | — | uc003yti.2 | A and B |
| AC016912.3 | novel transcript | OTTHUMT00000329731 | A and B |
| — | — | TCONS_00001962- | A and B |
| | | XLOC_000102 | |
| RNU6-649P | RNA, U6 small | ENST00000384463 | A and B |
| | nuclear 649, | | |
| | pseudogene | | |
| — | — | AK126681 | A and B |
| — | — | ENST00000541007 | A and B |
| — | — | DQ586768 | A and B |
| CERKL | ceramide kinase-like | NR_027689 | A and B |
| — | — | TCONS_l2_00030931- | A and B |
| | | XLOC_l2_015939 | |
| — | — | ENST00000384300 | A and B |
| FOXL1 | forkhead box L1 | NM_005250 | A and B |
| — | — | TCONS_00028198- | A and B |
| | | XLOC_013549 | |
| HLA-DRB1 | major | M35980 | A and B |
| | histocompatibility | | |
| | complex, class II, DR | | |
| | beta 1 | | |
| RNU6-870P | RNA, U6 small | ENST00000516994 | A and B |
| | nuclear 870, | | |
| | pseudogene | | |
| AP001631.10 | novel protein | OTTHUMT00000195568 | A and B |
| — | — | TCONS_00028994- | A and B |
| | | XLOC_013913 | |
| MIR323B | microRNA 323b | NR_036133 | A and B |
| LINC00622 | long intergenic non- | AK123168 | A and B |
| | protein coding RNA | | |
| | 622 | | |
| — | — | DQ598506 | A and B |
| LOC101928673 | uncharacterized | ENST00000367716 | A and B |
| | LOC101928673 | | |
| WWTR1-AS1 | WWTR1 antisense | NR_040250 | A and B |
| | RNA 1 | | |
| — | — | BC078139 | A and B |
| — | — | ENST00000440880 | A and B |
| — | — | ENST00000410690 | A and B |
| MIR548AC | microRNA 548ac | ENST00000408595 | A and B |
| — | — | TCONS_l2_00014953- | A and B |
| | | XLOC_l2_008316 | |
| LOC100132272 | uncharacterized | ENST00000378108 | A |
| | LOC100132272 | | |
| IGHV1-69 | immunoglobulin heavy | ENST00000390633 | A |
| | variable 1-69 | | |
| — | — | TCONS_00025738- | A |
| | | XLOC_012554 | |
| — | — | uc003tdl.1 | A |
| — | — | linc_luo_467 | A |
| SRMS | src-related kinase | NM_080823 | A |
| | lacking C-terminal | | |
| | regulatory tyrosine and | | |
| | N-terminal | | |
| | myristylation sites | | |
| — | — | ENST00000401253 | A |
| — | — | TCONS_00023596- | A |
| | | XLOC_011408 | |
| — | — | TCONS_00018405- | A |
| | | XLOC_008690 | |
| — | — | ENST00000557226 | A |
| AC009499.2 | putative novel | OTTHUMT00000325407 | A |
| | transcript | | |
| RNU6-907P | RNA, U6 small | ENST00000390924 | A |
| | nuclear 907, | | |
| | pseudogene | | |
| — | — | AF009276 | A |
| — | — | TCONS_00007659- | A |
| | | XLOC_003735 | |
| LOC643072 | uncharacterized | ENST00000418474 | A |
| | LOC643072 | | |
| RNU6-292P | RNA, U6 small | ENST00000384056 | A |
| | nuclear 292, | | |
| | pseudogene | | |
| — | — | ENST00000541344 | A |
| MIR129-2 | microRNA 129-2 | NR_029697 | A |
| DNLZ | DNL-type zinc finger | NM_001080849 | A |
| CD276 | CD276 molecule | AJ583696 | A |
| — | — | TCONS_l2_00001572- | A |
| | | XLOC_l2_001153 | |
| — | — | ENST00000536455 | A |
| — | — | ENST00000559825 | A |
| — | — | U29119 | A |
| — | — | TCONS_00010555- | A |
| | | XLOC_005082 | |
| HTR1D | 5-hydroxytryptamine | NM_000864 | A |
| | (serotonin) receptor | | |
| | 1D, G protein-coupled | | |
| — | — | AC002382 | A |
| LOC284632 | uncharacterized | BC033556 | A |
| | LOC284632 | | |
| AC003088.1 | novel transcript | OTTHUMT00000338092 | A |
| — | — | linc_luo_1995 | A |
| — | — | TCONS_l2_00031035- | A |
| | | XLOC_l2_015932 | |
| RP11-76G10.1 | novel transcript | OTTHUMT00000364997 | A |
| — | — | TCONS_00003485- | A |
| | | XLOC_002469 | |
| — | — | TCONS_00007384- | A |
| | | XLOC_003503 | |
| — | — | ENST00000515139 | A |
| — | — | TCONS_00026954- | A |
| | | XLOC_013012 | |
| — | — | ENST00000390161 | A |
| RP11-91A18.4 | putative novel | OTTHUMT00000023822 | A |
| | transcript | | |
| DGCR10 | DiGeorge syndrome | L77559 | A |
| | critical region gene 10 | | |
| | (non-protein coding) | | |
| — | — | ENST00000558785 | A |
| THY1 | Thy-1 cell surface | S59749 | A |
| | antigen | | |
| USP44 | ubiquitin specific | ENST00000547951 | A |
| | peptidase 44 | | |
| — | — | DQ590016 | A |
| — | — | OTTHUMT00000368425 | A |
| — | — | ENST00000362637 | A |
| — | — | ENST00000363682 | A |
| — | — | ENST00000364695 | A |
| — | — | TCONS_00000939- | A |
| | | XLOC_000191 | |
| MIR3130-1 | microRNA 3130-1 | NR_036077 | A |
| RP1-20N2.6 | novel transcript | OTTHUMT00000042524 | A |
| RNU6-525P | RNA, U6 small | ENST00000363685 | A |
| | nuclear 525, | | |
| | pseudogene | | |
| RP11-14N7.2 | novel transcript | OTTHUMT00000046024 | A |
| — | — | TCONS_00007468- | A |
| | | XLOC_003444 | |
| LINC01126 | long intergenic non- | NR_027251 | A |
| | protein coding RNA | | |
| | 1126 | | |
| RP11-137H2.4 | putative novel | OTTHUMT00000049090 | A |
| | transcript | | |
| — | — | AL080086 | A |
| RP11-400D2.3 | novel transcript | OTTHUMT00000365043 | A |
| — | — | uc021ysn.1 | A |
| — | — | linc_luo_331 | A |
| FGFBP1 | fibroblast growth | NM_005130 | A |
| | factor binding protein 1 | | |
| LINC00890 | long intergenic non- | NR_033974 | A |
| | protein coding RNA | | |
| | 890 | | |
| GAS6-AS1 | GAS6 antisense RNA 1 | NR_044995 | A |
| RP11-473O4.4 | putative novel | OTTHUMT00000380594 | A |
| | transcript | | |
| LOC100291666 | serologically defined | AF308290 | A |
| | breast cancer antigen | | |
| | NY-BR-40 | | |
| — | — | TCONS_00028426- | A |
| | | XLOC_013778 | |
| AC107057.1 | putative novel | OTTHUMT00000322559 | A |
| | transcript | | |
| — | — | TCONS_00000325- | A |
| | | XLOC_000443 | |
| KRTAP2-2 | keratin associated | NM_033032 | A |
| | protein 2-2 | | |
| — | — | TCONS_00000192- | A |
| | | XLOC_000173 | |
| LINC00106 | long intergenic non- | ENST00000430235 | A |
| | protein coding RNA | | |
| | 106 | | |
| RP11-10J21.5 | novel transcript | OTTHUMT00000378944 | A |
| ERI2 | ERI1 exoribonuclease | NM_001142725 | A |
| | family member 2 | | |
| ZDHHC24 | zinc finger, DHHC- | NM_207340 | A |
| | type containing 24 | | |
| SNORD97 | small nucleolar RNA, | NR_004403 | A |
| | C/D box 97 | | |
| MIR130A | microRNA 130a | NR_029673 | A |
| FAM90A25P | family with sequence | NR_036463 | A |
| | similarity 90, member | | |
| | A7 pseudogene | | |
| WISP1 | WNT1 inducible | NR_037944 | A |
| | signaling pathway | | |
| | protein 1 | | |
| — | — | AF075037 | A |
| RP11- | putative novel | OTTHUMT00000055264 | A |
| 229P13.22 | transcript | | |
| RNU6-937P | RNA, U6 small | ENST00000384325 | A |
| | nuclear 937, | | |
| | pseudogene | | |
| RNU2-56P | RNA, U2 small | ENST00000516826 | A |
| | nuclear 56, | | |
| | pseudogene | | |
| — | — | TCONS_l2_00003602- | A |
| | | XLOC_l2_002006 | |
| RP11- | putative novel | OTTHUMT00000320736 | A |
| 375H17.1 | transcript | | |
| — | — | ENST00000516734 | A |
| LOC729218 | uncharacterized | AK024248 | A |
| | LOC729218 | | |
| — | — | ENST00000410594 | A |
| TMCO2 | transmembrane and | NM_001008740 | A |
| | coiled-coil domains 2 | | |
| RP11-101E14.3 | novel transcript | OTTHUMT00000079228 | A |
| — | — | TCONS_00007906- | A |
| | | XLOC_004176 | |
| MNX1-AS1 | MNX1 antisense RNA | NR_038835 | A |
| | 1 (head to head) | | |
| CBX4 | chromobox homolog 4 | U94344 | A |
| — | — | TCONS_00012345- | A |
| | | XLOC_005899 | |
| DEFB123 | defensin, beta 123 | NM_153324 | A |
| — | — | DQ594725 | A |
| — | — | ENST00000408710 | A |
| — | — | TCONS_00025133- | A |
| | | XLOC_012382 | |
| — | — | TCONS_00019740- | A |
| | | XLOC_009534 | |
| FAM47B | family with sequence | NM_152631 | A |
| | similarity 47, member B | | |
| TFG | TRK-fused gene | NM_001007565 | A |
| AC012462.3 | novel transcript | OTTHUMT00000341267 | A |
| EPOR | erythropoietin receptor | NR_033663 | A |
| MIR338 | microRNA 338 | NR_029897 | A |
| — | — | CR613685 | A |
| DUX4L2 | double homeobox 4 | NM_001127386 | A |
| | like 2 | | |
| — | — | TCONS_00003325- | A |
| | | XLOC_002175 | |
| RP3-417O22.3 | novel transcript | OTTHUMT00000041565 | A |
| — | — | TCONS_00026485- | A |
| | | XLOC_012811 | |
| — | — | linc_luo_828 | A |
| — | — | TCONS_l2_00010598- | A |
| | | XLOC_l2_005691 | |
| 2-Sep | septin 2 | NM_001008491 | A |
| AC104135.3 | novel transcript | OTTHUMT00000328656 | A |
| MIR762 | microRNA 762 | NR_031576 | A |
| — | — | BC032027 | A |
| OR10AG1 | olfactory receptor, | NM_001005491 | A |
| | family 10, subfamily | | |
| | AG, member 1 | | |
| SPAM1 | sperm adhesion | L13779 | A |
| | molecule 1 (PH-20 | | |
| | hyaluronidase, zona | | |
| | pellucida binding) | | |
| — | — | TCONS_00012367- | A |
| | | XLOC_005932 | |
| — | — | uc003erl.1 | A |
| RP11-86A5.1 | novel transcript | OTTHUMT00000056119 | A |
| SNORD88A | small nucleolar RNA, | NR_003067 | A |
| | C/D box 88A | | |
| RP11-292F9.1 | novel transcript | OTTHUMT00000037029 | A |
| — | — | uc021ysa.1 | A |
| — | — | uc021sji.1 | A |
| — | — | L38562 | A |
| LOC101060602 | multidrug and toxin | ENST00000420951 | A |
| | extrusion protein 2-like | | |
| RNU6-1282P | RNA, U6 small | ENST00000516735 | A |
| | nuclear 1282, | | |
| | pseudogene | | |
| LINC00261 | long intergenic non- | ENST00000420070 | A |
| | protein coding RNA | | |
| | 261 | | |
| — | — | AK130541 | A |
| RP5-983L19.2 | novel transcript | OTTHUMT00000317428 | A |
| NAGLU | N- | NM_000263 | A |
| | acetylglucosaminidase, | | |
| | alpha | | |
| — | — | TCONS_00013447- | A |
| | | XLOC_006100 | |
| TAB1 | TGF-beta activated | EF036484 | A |
| | kinase 1/MAP3K7 | | |
| | binding protein 1 | | |
| — | — | CR600243 | A |
| — | — | TCONS_00003876- | A |
| | | XLOC_001676 | |
| — | — | AF086424 | A |
| — | — | uc002dam.1 | A |
| COPS7A | COP9 signalosome | NM_001164093 | A |
| | subunit 7A | | |
| RASSF3 | Ras association | NM_178169 | A |
| | (RalGDS/AF-6) | | |
| | domain family member 3 | | |
| RNA5SP89 | RNA, 5S ribosomal | ENST00000410300 | A |
| | pseudogene 89 | | |
| — | — | BC126309 | A |
| — | — | TCONS_00020943- | A |
| | | XLOC_010213 | |
| — | — | TCONS_00018253- | A |
| | | XLOC_008530 | |
| RNU6-54P | RNA, U6 small | ENST00000365563 | A |
| | nuclear 54, | | |
| | pseudogene | | |
| — | — | TCONS_00015772- | A |
| | | XLOC_007602 | |
| RNU6-767P | RNA, U6 small | ENST00000384132 | A |
| | nuclear 767, | | |
| | pseudogene | | |
| HOXC-AS2 | HOXC cluster | ENST00000513533 | A |
| | antisense RNA 2 | | |
| — | — | ENST00000410631 | A |
| — | — | uc022api.1 | A |
| — | — | ENST00000384553 | A |
| — | — | TCONS_l2_00006293- | A |
| | | XLOC_l2_003401 | |
| — | — | TCONS_l2_00007350- | A |
| | | XLOC_l2_003606 | |
| — | — | uc021wbs.1 | A |
| — | — | TCONS_00029593- | A |
| | | XLOC_014237 | |
| — | — | TCONS_00015021- | A |
| | | XLOC_007095 | |
| NKX2-5 | NK2 homeobox 5 | NM_001166175 | A |
| — | — | BC043266 | A |
| C22orf31 | chromosome 22 open | NM_015370 | A |
| | reading frame 31 | | |
| — | — | TCONS_00011591- | A |
| | | XLOC_005870 | |
| OR5E1P | olfactory receptor, | AF309699 | A |
| | family 5, subfamily E, | | |
| | member 1 pseudogene | | |
| — | — | TCONS_00021206- | A |
| | | XLOC_009869 | |
| — | — | TCONS_00026281- | A |
| | | XLOC_012627 | |
| — | — | TCONS_00003099- | A |
| | | XLOC_001847 | |
| MIR3648-1 | microRNA 3648-1 | NR_037421 | A |
| — | — | AK127874 | A |
| RP11-15B24.4 | putative novel | OTTHUMT00000052822 | A |
| | transcript | | |
| — | — | ENST00000543061 | A |
| — | — | AK022971 | A |
| — | — | linc_luo_993 | A |
| MIR572 | microRNA 572 | NR_030298 | A |
| RP11-402P6.7 | putative novel | OTTHUMT00000058868 | A |
| | transcript | | |
| RP11-402P6.11 | putative novel | OTTHUMT00000057168 | A |
| | transcript | | |
| STK19 | serine/threonine kinase | NR_026717 | A |
| | 19 | | |
| LINC00238 | long intergenic non- | BC056671 | A |
| | protein coding RNA | | |
| | 238 | | |
| — | — | AJ508601 | A |
| AP006216.5 | putative novel | OTTHUMT00000106282 | A |
| | transcript | | |
| ROGDI | rogdi homolog | BC113944 | A |
| | (Drosophila) | | |
| RP11-484O2.1 | novel transcript | OTTHUMT00000359983 | A |
| TRBV7-3 | T cell receptor beta | ENST00000390361 | A |
| | variable 7-3 | | |
| — | — | DQ594696 | A |
| SLC10A5 | solute carrier family | NM_001010893 | A |
| | 10, member 5 | | |
| TNK2-AS1 | TNK2 antisense RNA 1 | ENST00000458180 | A |
| — | — | ENST00000560237 | A |
| LOC100132686 | uncharacterized | BC020894 | A |
| | LOC100132686 | | |
| RP11-893F2.5 | novel transcript | OTTHUMT00000367043 | A |
| — | — | ENST00000553318 | A |
| BOK-AS1 | BOK antisense RNA 1 | NR_033346 | A |
| — | — | ENST00000525424 | A |
| — | — | TCONS_00001418- | A |
| | | XLOC_000737 | |
| RNU6-986P | RNA, U6 small | ENST00000363133 | A |
| | nuclear 986, | | |
| | pseudogene | | |
| CCDC88C | coiled-coil domain | BC127900 | A |
| | containing 88C | | |
| MYADML2 | myeloid-associated | NM_001145113 | A |
| | differentiation marker- | | |
| | like 2 | | |
| CXorf21 | chromosome X open | NM_025159 | A |
| | reading frame 21 | | |
| — | — | TCONS_l2_00003037- | A |
| | | XLOC_l2_001585 | |
| CTD- | novel transcript | OTTHUMT00000374703 | A |
| 3118D11.3 | | | |
| RNU6-811P | RNA, U6 small | ENST00000384069 | A |
| | nuclear 811, | | |
| | pseudogene | | |
| LOC100507477 | uncharacterized | ENST00000418834 | A |
| | LOC100507477 | | |
| MIR1302-1 | microRNA 1302-1 | ENST00000408633 | A |
| RP11-51B13.1 | putative novel protein | OTTHUMT00000045439 | A |
| C1orf68 | chromosome 1 open | AF005081 | A |
| | reading frame 68 | | |
| RNU6-1020P | RNA, U6 small | ENST00000363684 | A |
| | nuclear 1020, | | |
| | pseudogene | | |
| LOC101927619 | uncharacterized | AK096499 | A |
| | LOC101927619 | | |
| — | — | TCONS_00014983- | A |
| | | XLOC_007064 | |
| — | — | ENST00000526906 | A |
| SLC25A10 | solute carrier family 25 | NM_012140 | A |
| | (mitochondrial carrier; | | |
| | dicarboxylate | | |
| | transporter), member | | |
| | 10 | | |
| CMC1 | C—x(9)—C motif | CR749370 | A |
| | containing 1 | | |
| RP11-577B7.1 | novel transcript | OTTHUMT00000367011 | A |
| — | — | ENST00000542627 | A |
| — | — | AK026734 | A |
| SURF2 | surfeit 2 | NM_017503 | A |
| — | — | ENST00000362620 | A |
| RP11-535C7.1 | putative novel | OTTHUMT00000361472 | A |
| | transcript | | |
| — | — | TCONS_l2_00024447- | A |
| | | XLOC_l2_012741 | |
| RP11-889D3.2 | novel transcript | OTTHUMT00000350794 | A |
| RP3-413H6.2 | novel transcript | OTTHUMT00000039866 | A |
| MIR3938 | microRNA 3938 | NR_037502 | A |
| OGG1 | 8-oxoguanine DNA | AB037880 | A |
| | glycosylase | | |
| RP13- | novel transcript, | OTTHUMT00000343245 | A |
| 766D20.2 | antisense to ACTG1 | | |
| — | — | ENST00000553990 | A |
| KRTAP21-1 | keratin associated | ENST00000416521 | A |
| | protein 21-1 | | |
| SNORA78 | small nucleolar RNA, | BC028232 | A |
| | H/ACA box 78 | | |
| RP4-781K5.4 | novel transcript | OTTHUMT00000092701 | A |
| — | — | TCONS_00020467- | A |
| | | XLOC_009800 | |
| AZGP1P1 | alpha-2-glycoprotein 1, | NR_036679 | A |
| | zinc-binding | | |
| | pseudogene 1 | | |
| RP4-742C19.12 | apolipoprotein B | OTTHUMT00000321691 | A |
| | mRNA editing | | |
| | enzyme, catalytic | | |
| | polypeptide-like 3 | | |
| | (APOBEC3) family | | |
| | pseudogene | | |
| AC022816.2 | novel transcript | OTTHUMT00000130000 | A |
| RNU6-38P | RNA, U6 small | ENST00000384085 | A |
| | nuclear 38, | | |
| | pseudogene | | |
| — | — | uc002zvv.2 | A |
| — | — | TCONS_00013525- | A |
| | | XLOC_006166 | |
| MIR4324 | microRNA 4324 | NR_036209 | A |
| RP11-65D24.2 | novel protein | OTTHUMT00000045814 | A |
| — | — | TCONS_00015671- | A |
| | | XLOC_007357 | |
| — | — | ENST00000516667 | A |
| — | — | DQ590525 | A |
| RP11- | putative novel | OTTHUMT00000026685 | A |
| 415A20.1 | transcript | | |
| KB-1930G5.3 | putative novel | OTTHUMT00000380525 | A |
| | transcript | | |
| — | — | AK022165 | A |
| LOC100505921 | uncharacterized | ENST00000451066 | A |
| | LOC100505921 | | |
| — | — | TCONS_00005647- | A |
| | | XLOC_002908 | |
| — | — | TCONS_00025884- | A |
| | | XLOC_012161 | |
| — | — | ENST00000411845 | A |
| — | — | TCONS_l2_00019027- | A |
| | | XLOC_l2_010018 | |
| HMX2 | H6 family homeobox 2 | NM_005519 | A |
| — | — | TCONS_00019770- | A |
| | | XLOC_009564 | |
| — | — | TCONS_00017098- | A |
| | | XLOC_008251 | |
| RP11- | novel transcript | OTTHUMT00000056135 | A |
| 268G12.3 | | | |
| — | — | TCONS_00020560- | A |
| | | XLOC_009876 | |
| — | — | ENST00000410769 | A |
| FAM72D | family with sequence | NM_207418 | A |
| | similarity 72, member D | | |
| PCDHB18 | protocadherin beta 18 | NR_001281 | A |
| | pseudogene | | |
| RNU6-461P | RNA, U6 small | ENST00000364195 | A |
| | nuclear 461, | | |
| | pseudogene | | |
| TAS2R39 | taste receptor, type 2, | NM_176881 | A |
| | member 39 | | |
| — | — | TCONS_00023434- | A |
| | | XLOC_011275 | |
| — | — | TCONS_00017953- | A |
| | | XLOC_008779 | |
| RNU6-1095P | RNA, U6 small | ENST00000516148 | A |
| | nuclear 1095, | | |
| | pseudogene | | |
| — | — | AF087983 | A |
| LINC00662 | long intergenic non- | NR_027301 | A |
| | protein coding RNA | | |
| | 662 | | |
| — | — | D16470 | A |
| LOC100289511 | uncharacterized | NR_029378 | A |
| | LOC100289511 | | |
| CCDC87 | coiled-coil domain | NM_018219 | A |
| | containing 87 | | |
| RNU6-1260P | RNA, U6 small | ENST00000362944 | A |
| | nuclear 1260, | | |
| | pseudogene | | |
| — | — | ENST00000459492 | A |
| — | — | ENST00000420972 | A |
| — | — | L43846 | A |
| PCYT2 | phosphate | NM_001184917 | A |
| | cytidylyltransferase 2, | | |
| | ethanolamine | | |
| ZNF853 | zinc finger protein 853 | NM_017560 | A |
| MIR548A3 | microRNA 548a-3 | NR_030330 | A |
| RP3-410C9.1 | novel transcript | OTTHUMT00000078483 | A |
| — | — | TCONS_l2_00005790- | A |
| | | XLOC_l2_003070 | |
| MIR676 | microRNA 676 | NR_037494 | A |
| — | — | ENST00000558375 | A |
| MIR548A2 | microRNA 548a-2 | ENST00000384956 | A |
| — | — | ENST00000391069 | A |
| RNU6-462P | RNA, U6 small | ENST00000362659 | A |
| | nuclear 462, | | |
| | pseudogene | | |
| — | — | TCONS_00000575- | A |
| | | XLOC_000921 | |
| — | — | ENST00000429933 | A |
| — | — | TCONS_00019786- | A |
| | | XLOC_009584 | |
| — | — | TCONS_l2_00019084- | A |
| | | XLOC_l2_010061 | |
| — | — | 342955 | A |
| PPM1A | protein phosphatase, | AY236965 | A |
| | Mg2+/Mn2+ | | |
| | dependent, 1A | | |
| — | — | BC061594 | A |
| RP1-212P9.2 | putative novel | OTTHUMT00000010343 | A |
| | transcript | | |
| AC092660.1 | novel transcript | OTTHUMT00000328311 | A |
| RP4-710M16.2 | novel transcript | OTTHUMT00000022253 | A |
| DUX4L2 | double homeobox 4 | NM_001127386 | A |
| | like 2 | | |
| DUX4L2 | double homeobox 4 | NM_001127386 | A |
| | like 2 | | |
| RP5-1010E17.2 | novel transcript | OTTHUMT00000259284 | A |
| KIF11 | kinesin family member | BC050667 | A |
| | 11 | | |
| RNU6-1092P | RNA, U6 small | ENST00000516955 | A |
| | nuclear 1092, | | |
| | pseudogene | | |
| RNU6-684P | RNA, U6 small | ENST00000410829 | A |
| | nuclear 684, | | |
| | pseudogene | | |

### Compositions

Provided herein are colorectal cancer biomarker genes and panels of colorectal cancer biomarker genes for use in diagnosis of colorectal cancer. A biomarker is generally a characteristic that can be objectively measured and quantified and used to evaluate a biological process, for example, colorectal cancer development, progression, remission, and recurrence. Biomarkers can take many forms including, nucleic acids, polypeptides, metabolites, or physical or physiological parameters.

We may refer to any of the genes listed in Table 1 as colorectal cancer biomarker genes. The colorectal cancer biomarker genes of the invention include nucleic acid sequences, for example, total RNA, total DNA, mRNA, tRNA, rRNA, ncRNA, smRNA, and snoRNA, whose measured expression levels are different from, i.e., increased or decreased, in a subject having colorectal cancer or who is at risk for colorectal cancer, relative to the measured expression levels of the same markers in a healthy subject.

Nucleic Acids.

We may use the terms “nucleic acid” and “polynucleotide” interchangeably to refer to both RNA and DNA, including cDNA, genomic DNA, synthetic DNA, and DNA (or RNA) containing nucleic acid analogs, any of which may encode a polypeptide of the invention and all of which are encompassed by the invention. Polynucleotides can have essentially any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode a fragment of a biomarker selected from Table 1 or a biologically active variant thereof.

An “isolated” nucleic acid can be, for example, a DNA molecule or a fragment thereof, provided that at least one of the nucleic acid sequences normally found immediately flanking that DNA molecule in a genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced in a variety of ways. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a portion of biomarker DNA selected from Table 1.

Two nucleic acids or the polypeptides they encode may be described as having a certain degree of identity to one another. For example, a colorectal cancer biomarker gene selected from Table 1 and a biologically active variant thereof may be described as exhibiting a certain degree of identity. Alignments may be assembled by locating short sequences in the Protein Information Research (PIR) site (http://pir.georgetown.edu), followed by analysis with the “short nearly identical sequences” Basic Local Alignment Search Tool (BLAST) algorithm on the NCBI website (http://www.ncbi.nlm.nih.gov/blast).

As used herein, the term “percent sequence identity” refers to the degree of identity between any given query sequence and a subject sequence. For example, a colorectal cancer biomarker gene sequence listed in Table 1 can be the query sequence and a fragment of a colorectal cancer biomarker gene sequence listed in Table 1 can be the subject sequence. Similarly, a fragment of a colorectal cancer biomarker gene sequence listed in Table 1 can be the query sequence and a biologically active variant thereof can be the subject sequence.

To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment).

ClustalW calculates the best match between a query and one or more subject sequences and aligns them so that identities, similarities and differences can be determined. Gaps of one or more residues can be inserted into a query sequence, a subject sequence, or both, to maximize sequence alignments. For fast pair wise alignment of nucleic acid sequences, the following default parameters are used: word size: 2; window size: 4; scoring method: percentage; number of top diagonals: 4; and gap penalty: 5. For multiple alignments of nucleic acid sequences, the following parameters are used: gap opening penalty: 10.0; gap extension penalty: 5.0; and weight transitions: yes. For fast pair wise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The nucleic acids and polypeptides described herein may be referred to as “exogenous”. The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the native sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

Nucleic acids of the invention, that is, nucleic acids having a nucleotide sequence of any one of the colorectal cancer biomarkers listed in Table 1, can include nucleic acids sequences that are at least about 70%, at least about 75%, at least about 80%, at least about 85%, at least about 90%, at least about 95%, at least about 99% identical to the sequences provided by the accession numbers listed in Table 1.

A nucleic acid, for example, an oligonucleotide (e.g., a probe or a primer) that is specific for a target nucleic acid will hybridize to the target nucleic acid under suitable conditions. We may refer to hybridization or hybridizing as the process by which an oligonucleotide single strand anneals with a complementary strand through base pairing under defined hybridization conditions. It is a specific, i.e., non-random, interaction between two complementary polynucleotides. Hybridization and the strength of hybridization (i.e., the strength of the association between the nucleic acids) is influenced by such factors as the degree of complementary between the nucleic acids, stringency of the conditions involved, and the melting temperature (Tm) of the formed hybrid. The hybridization products can be duplexes or triplexes formed with targets in solution or on solid supports.

In some embodiments, the nucleic acids can include short nucleic acid sequences useful for analysis and quantification of the colorectal cancer biomarker genes listed in Table 1. Such isolated nucleic acids can be oligonucleotide primers. In general, an oligonucleotide primer is an oligonucleotide complementary to a target nucleotide sequence, for example, the nucleotide sequence of any of the colorectal cancer biomarker genes listed in Table 1, that can serve as a starting point for DNA synthesis by the addition of nucleotides to the 3′ end of the primer in the presence of a DNA or RNA polymerase. The 3′ nucleotide of the primer should generally be identical to the target sequence at a corresponding nucleotide position for optimal extension and/or amplification. Primers can take many forms, including for example, peptide nucleic acid primers, locked nucleic acid primers, unlocked nucleic acid primers, and/or phosphorothioate modified primers. In some embodiments, a forward primer can be a primer that is complementary to the anti-sense strand of dsDNA and a reverse primer can be a primer that is complementary to the sense-strand of dsDNA. We may also refer to primer pairs. In some embodiments, a 5′ target primer pair can be a primer pair that includes at least one forward primer and at least one reverse primer that amplifies the 5′ region of a target nucleotide sequence. In some embodiments, a 3′ target primer pair can be a primer pair at least one forward primer and at least one reverse primer that amplifies the 3′ region of a target nucleotide sequence. In some embodiments the primer can include a detectable label, as discussed below.

Oligonucleotide primers provided herein are useful for amplification of any of the colorectal cancer biomarker gene sequences listed in Table 1. In some embodiments, oligonucleotide primers can be complementary to two or more of the colorectal cancer biomarker genes disclosed herein, for example, the colorectal cancer biomarker genes listed in Table 1. The primer length can vary depending upon the nucleotide base sequence and composition of the particular nucleic acid sequence of the probe and the specific method for which the probe is used. In general, useful primer lengths can be about 8, 9, 10, 11, 12, 13, 14, 15, 16, 17, 18, 19, 20, 21, 22, 23, 24, 25, 26, 27, 28, 29, 30 nucleotide bases. Useful primer lengths can range from 8 nucleotide bases to about 60 nucleotide bases; from about 12 nucleotide bases to about 50 nucleotide bases; from about 12 nucleotide bases to about 45 nucleotide bases; from about 12 nucleotide bases to about 40 nucleotide bases; from about 12 nucleotide bases to about 35 nucleotide bases; from about 15 nucleotide bases to about 40 nucleotide bases; from about 15 nucleotide bases to about 35 nucleotide bases; from about 18 nucleotide bases to about 50 nucleotide bases; from about 18 nucleotide bases to about 40 nucleotide bases; from about 18 nucleotide bases to about 35 nucleotide bases; from about 18 nucleotide bases to about 30 nucleotide bases; from about 20 nucleotide bases to about 30 nucleotide bases; from about 20 nucleotide bases to about 25 nucleotide bases.

Also provided are probes, that is, isolated nucleic acid fragments that selectively bind to and are complementary to any of the colorectal cancer biomarker gene sequences listed in Table 1. Probes can be oligonucleotides or polynucleotides, DNA or RNA, single- or double-stranded, and natural or modified, either in the nucleotide bases or in the backbone. Probes can be produced by a variety of methods including chemical or enzymatic synthesis.

The probe length can vary depending upon the nucleotide base sequence and composition of the particular nucleic acid sequence of the probe and the specific method for which the probe is used. In general, useful probe lengths can be about 8, 9, 10, 11, 12, 13, 14, 15, 16, 17, 18, 19, 20, 21, 22, 23, 24, 25, 26, 27, 28, 29, 30, 35, 40, 50, 55, 60, 65, 70, 75, 80, 85, 90, 100, 110, 120, 140, 150, 175, or 200 nucleotide bases. In general, useful probe lengths will range from about 8 to about 200 nucleotide bases; from about 12 to about 175 nucleotide bases; from about 15 to about 150 nucleotide bases; from about 15 to about 100 nucleotide bases from about 15 to about 75 nucleotide bases; from about 15 to about 60 nucleotide bases; from about 20 to about 100 nucleotide bases; from about 20 to about 75 nucleotide bases; from about 20 to about 60 nucleotide bases; from about 20 to about 50 nucleotide bases in length. In some embodiments the probe set can comprise probes directed to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100, 125, 150, 175, 200, 225, 250, 275, 300, 325, 350, 375, 400, 425, 450, 475, 500, 525, 550, 575 or more, or all, of the colorectal cancer biomarker genes in Table 1.

The primers and probes disclosed herein can be detectably labeled. A label can be a molecular moiety or compound that can be detected or lead to a detectable response, which may be joined directly or indirectly to a nucleic acid. Direct labeling may use bonds or interactions to link label and probe, which includes covalent bonds, non-covalent interactions (hydrogen bonds, hydrophobic and ionic interactions), or chelates or coordination complexes. Indirect labeling may use a bridging moiety or linker (e.g. antibody, oligomer, or other compound), which is directly or indirectly labeled, which may amplify a signal. Labels include any detectable moiety, e.g., radionuclide, ligand such as biotin or avidin, enzyme, enzyme substrate, reactive group, chromophore (detectable dye, particle, or bead), fluorophore, or luminescent compound (bioluminescent, phosphorescent, or chemiluminescent label). Labels can be detectable in a homogeneous assay in which bound labeled probe in a mixture exhibits a detectable change compared to that of unbound labeled probe, e.g., stability or differential degradation, without requiring physical separation of bound from unbound forms.

Suitable detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). As discussed above, coupling of the one or more ligand motifs and/or ligands to the detectable label may be direct or indirect. Detection may be in situ, in vivo, in vitro on a tissue section or in solution, etc.

In some embodiments, the methods include the use of alkaline phosphatase conjugated polynucleotide probes. When an alkaline phosphatase (AP)-conjugated polynucleotide probe is used, following sequential addition of an appropriate substrate such as fast blue or fast red substrate, AP breaks down the substrate to form a precipitate that allows in-situ detection of the specific target RNA molecule. Alkaline phosphatase may be used with a number of substrates, e.g., fast blue, fast red, or 5-Bromo-4-chloro-3-indolyl-phosphate (BCIP). See, e.g., as described generally in U.S. Pat. Nos. 5,780,277 and 7,033,758.

In some embodiments, the fluorophore-conjugates probes can be fluorescent dye conjugated label probes, or utilize other enzymatic approaches besides alkaline phosphatase for a chromogenic detection route, such as the use of horseradish peroxidase conjugated probes with substrates like 3,3′-Diaminobenzidine (DAB).

The fluorescent dyes used in the conjugated label probes may typically be divided into families, such as fluorescein and its derivatives; rhodamine and its derivatives; cyanine and its derivatives; coumarin and its derivatives; Cascade Blue™ and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa Fluor®-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine™), carboxy tetramethylrhodamine (TAMRA™), carboxy-X-rhodamine (ROX™), LIZ™, VIC™, NED™, PET™, SYBR, PicoGreen, RiboGreen, and the like. Descriptions of fluorophores and their use, can be found in, among other places, R. Haugland, Handbook of Fluorescent Probes and Research Products, 9th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va. Near-infrared dyes are expressly within the intended meaning of the terms fluorophore and fluorescent reporter group.

In some embodiments, the probes and probe sets can be configured as a gene array. A gene array, also known as a microarray or a gene chip, is an ordered array of nucleic acids that allows parallel analysis of complex biological samples. Typically a gene array includes probes that are attached to a solid substrate, for example a microchip, a glass slide, or a bead. The attachment generally involves a chemical coupling resulting in a covalent bond between the substrate and the probe. The number of probes in an array can vary, but each probe is fixed to a specific addressable location on the array or microchip. In some embodiments, the probes can be about 18 nucleotide bases, about 20 nucleotide bases, about 25 nucleotide bases, about 30 nucleotide bases, about 35 nucleotide bases, or about 40 nucleotide bases in length. In some embodiments the probe set comprises probes directed to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100, 125, 150, or more, or all, of the colorectal cancer biomarker genes in Table 1. For example, the probe set can include probes directed to the colorectal cancer biomarker genes in Panel A, Panel B, Panel C, Panel D, Panel E, or subsets of the colorectal cancer biomarkers in Panel A, Panel B, Panel C, Panel D, Panel E. The probe sets can be incorporated into high-density arrays comprising 5,000, 10,000, 20,000, 50,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000 or more different probes.

Methods of gene array synthesis can vary. Exemplary methods include synthesis of the probes followed by deposition onto the array surface by “spotting,” in situ synthesis, using for example, photolithography, or electrochemistry on microelectrode arrays.

### Methods

The compositions disclosed herein are generally and variously useful for the detection, diagnosis and treatment of colorectal cancer. Methods of detection can include measuring the expression level in a stool sample of two or more colorectal cancer biomarkers selected from the biomarkers listed in any of Table 1 and comparing the measured expression level of the two or more colorectal cancer biomarker genes in the sample with the measured expression level of two or more colorectal cancer biomarker genes in a control sample. A difference in the measured expression level of two or more colorectal cancer biomarker genes in a patient's sample relative to the measured expression level of the two or more colorectal cancer biomarker genes in a control sample is an indication that the patient has or is at risk for colorectal cancer. These methods can further include the step of identifying a subject (e.g., a patient and, more specifically, a human patient) who has colorectal cancer or who is at risk for colorectal cancer.

Colorectal cancer can include any form of colorectal cancer. Colorectal cancer typically begins as a growth, termed a polyp, in the inner lining of the colon or rectum. Colorectal polyps are generally divided into two categories: adenomatous polyps, also called adenomas; and hyperplastic and inflammatory polyps. Adenomatous polyps can give rise to colorectal cancer. The most common form of colorectal cancer, adenocarcinoma, originates in the intestinal gland cells that line the inside of the colon and/or rectum. Adenocarcinomas can include tubular adenocarcinomas, which are glandular cancers on a pedunculated stalk, and villous adenocarcinomas, which are glandular cancers that lie flat on the surface of the colon. Other colorectal cancers are distinguished by their tissue of origin. These include gastrointestinal stromal tumors (GIST), which arise from the interstitial cells of Cajal; primary colorectal lymphomas, which arise from hematologic cells; leiomyosarcomas, which are sarcomas arising from connective tissue or smooth muscle; melanomas, which arise from melanocytes: squamous cell carcinomas which arise from stratified squamous epithelial tissue and are confined to the rectum; and mucinous carcinomas, which are epithelial cancers generally associated with poor prognosis.

Symptoms of colorectal cancer can include, but are not limited to, a change in bowel habits, including diarrhea or constipation or a change in the consistency of the stool lasting longer than four weeks, rectal bleeding or blood in the stool, persistent abdominal discomfort such as cramps, gas or pain, a feeling that the bowel does not empty completely, weakness or fatigue, and unexplained weight loss. Patients suspected of having colorectal cancer may receive peripheral blood tests, including a complete blood count (CBC), a fecal occult blood test (FOBT), a liver function analysis, and a fecal immunochemical test for analysis of certain tumor markers, for example carcinoembryonic antigen (CEA) and CA19-9. Colorectal cancer is often diagnosed based on colonoscopy. During colonoscopy, any polyps that are noted are removed, biopsied and analyzed to determine whether the polyp contains colorectal cancer cells or cells that have undergone a precancerous change. Each one of the specific cancers listed above can look different when viewed through an endoscope. Villous adenomas melanomas, and squamous cell carcinomas are typically flat or sessile, whereas tubular adenomas, lymphomas, leiomyosarcomas and GIST tumors are typically pedunculated. However, flat and sessile adenomas can be missed by gastroenterologists during colonoscopies. Biopsy samples can be subjected to further analysis based on genetic changes of particular genes or microsatellite instability.

Other diagnostic methods can include, sigmoidoscopy, imaging tests, for example, computed tomography (CT or CAT) scans; ultrasound, for example abdominal, endorectal or intraoperative ultrasound, magnetic resonance imaging (MRI) scans, for example endorectal MRI. Other tests such as angiography and chest x-rays can be carried out to determine whether a colorectal cancer has metastasized.

A variety of methods for staging colorectal cancer have been developed. The most commonly used system, the TNM system is based on three factors: 1) the distance that the primary tumor (T) has grown into the wall of the intestine and nearby areas; 2) whether the tumor has spread to nearby regional lymph nodes (N); 3) whether the cancer has metastasized to other organs (M). Other methods of staging include Dukes staging and the Astler-Coller classification.

The TNM system provides a four-stage classification of colorectal cancer. In Stage 1 (T1) colorectal cancer, the tumor has grown into the layers of the colon wall, but has not spread outside the colon wall or into lymph nodes. If the cancer is part of a tubular adenoma polyp, then simple excision is performed and the patient can continue to receive routine testing for future cancer development. If the cancer is high grade or part of a flat/sessile polyp, more surgery might be required and larger margins will be taken; this might include partial colectomy where a section of the colon is resected. In Stage 2 (T2) colorectal cancer, the tumor has grown into the wall of the colon and potentially into nearby tissue but has not spread to nearby lymph nodes. Surgical removal of the tumor and a partial colectomy is generally performed. Adjunct therapy, for example, chemotherapy with agents such as 5-fluorouracil, leucovorin, or capecitabine, may be administered. Such tumors are unlikely to recur, but increased screening of the patient is generally needed. In Stage 3 (T3) colorectal cancer, the tumor has spread to nearby lymph nodes, but not to other parts of the body. Surgery to remove the section of the colon and all affected lymph nodes will be required. Chemotherapy, with agents such as 5-fluorouracil, leucovorin, oxaliplatin, or capecitabine combined with oxaliplatin is typically recommended. Radiation therapy may also be used depending on the age of the patient and aggressive nature of the tumor. In Stage 4 (T4) colorectal cancer, the tumor has spread from the colon to distant organs through the blood. Colorectal cancer most frequently metastasizes to the liver, lungs and/or peritoneum. Surgery is unlikely to cure these cancers and chemotherapy and or radiation are generally needed to improve survival rates.

The methods disclosed herein are generally useful for diagnosis and treatment of colorectal cancer. The level of two or more colorectal cancer biomarker genes is measured in a biological sample, that is a sample from a subject. The subject can be a patient having one or more of the symptoms described above that would indicate the patient is at risk for colorectal cancer. The subject can also be a patient having no symptoms, but who may be at risk for colorectal cancer based on age (for example, above age 50), family history, obesity, diet, alcohol consumption, tobacco use, previous diagnosis of colorectal polyps, race and ethnic background, inflammatory bowel disease, and genetic syndromes, such as familial adenomatous polyposis, Gardner syndrome, Lynch syndrome, Turcot syndrome, Peutz-Jeghers syndrome, and MUTYH-associated polyposis, associated with higher risk of colorectal cancer. The methods disclosed herein are also useful for monitoring a patient who has previously been diagnosed and treated for colorectal cancer in order to monitor remission and detect cancer recurrence.

A biological sample can be a sample that contains cells or other cellular material from which nucleic acids or other analytes can be obtained. A biological sample can be a stool sample provided by the subject. The stool sample can be obtained from a subject immediately following defecation. In some embodiments, the stool sample can be obtained from the subject following a procedure, such as an enema, to alleviate constipation, a condition often associated with colorectal cancer. In some embodiments, a stabilizing agent, for example a buffer or preservative, can be added to the stool sample following collection. The stool sample can be tested immediately. Alternatively, the stool sample can be collected and stored refrigerated (for example, at 4° C. or frozen, for example, at 0° C., −20° C. or −80° C. prior to testing.

Nucleic acids can be extracted from the biological sample, for example a stool sample, prior to analysis. Within the colon, there are about 10¹² bacterial cells per gram of intestinal content. This colonic microflora includes between 300-1000 species. A stool or fecal sample is a complex macromolecular mixture that includes not only human cells, but microbes, including bacteria and any gastrointestinal parasites, indigestible unabsorbed food residues, secretions from intestinal cells, and excreted material such as mucous and pigments. Normal stool is made up of about 75% water and 25% solid matter. Bacteria make up about 60% of the total dry mass of feces. The high bacterial load can contribute to an unfavorable signal-to-noise ratio for the detection of human sequences from a stool sample. In some embodiments, a stool sample can be processed to enrich for human nucleic acids.

Useful methods for isolation of nucleic acids from a stool sample that are enriched for human nucleic acids are provided herein. The method can include disrupting the stool sample with zirconium/silica beads and buffer. The sample can be subjected to vortexing, shaking, stirring, rotation, or other method of agitation sufficient to disperse the solids and the stool bacteria. The temperature at which the agitation and centrifugation steps are carried out can vary, for example, from about 4° C. to about 20° C., from about 4° C. to about 15° C., from about 4° C. to about 10° C., from about 4° C. to about 6° C. Following disruption, the sample can be subjected to one or more rounds of centrifugation. In some embodiments, the disruption step and the centrifugation can be repeated one, two, three, or more additional times. Commercially available reagents, for example Nuclisens® EasyMag® reagents can be used for stool disruption, washing, and cell lysis. Lysis buffer can also be to lyse the human cells. The lysate can be further centrifuged and the supernatant used for input into an automated RNA isolation machine, for example EasyMag® instrument. In some embodiments, the extracted nucleic acids can be treated with DNase to clear the solution of DNA. Other methods can be used including mechanical or enzymatic cell disruption followed by a solid phase method such as column chromatography or extraction with organic solvents, for example, phenol-chloroform or thiocyanate-phenol-chloroform extraction. In some embodiments, the nucleic acid can be extracted onto a functionalized bead. In some embodiments, the functionalized bead can further comprise a magnetic core (“magnetic bead.”) In some embodiments, the functionalized bead can include a surface functionalized with a charged moiety. The charged moiety can be selected from: amine, carboxylic acid, carboxylate, quaternary amine, sulfate, sulfonate, or phosphate.

The levels of the colorectal cancer markers can be evaluated using a variety of methods. Expression levels can be determined either at the nucleic acid, for example, the RNA level or at the polypeptide level. RNA expression can encompass expression of total RNA, mRNA, tRNA, rRNA, ncRNA, smRNA, miRNA, and snoRNA. Expression at the RNA level can be measured directly or indirectly by measuring levels of cDNA corresponding to the relevant RNA. Alternatively or in addition, polypeptides encoded by the RNA, RNA regulators of the genes encoding the relevant transcription factors, and levels of the transcription factor polypeptides can also be assayed. Methods for determining gene expression at the mRNA level include, for example, microarray analysis, serial analysis of gene expression (SAGE), RT-PCR, blotting, hybridization based on digital barcode quantification assays, multiplex RT-PCR, digital drop PCR (ddPCR), NanoDrop spectrophotometers, qRT-PCR, qPCR, UV spectroscopy, RNA sequencing, next-generation sequencing, lysate based hybridization assays utilizing branched DNA signal amplification such as the QuantiGene 2.0 Single Plex, and branched DNA analysis methods. Digital barcode quantification assays can include the BeadArray (Illumina), the xMAP systems (Luminex), the nCounter (Nanostring), the High Throughput Genomics (HTG) molecular, BioMark (Fluidigm), or the Wafergen microarray. Assays can include DASL (Illumina), RNA-Seq (Illumina), TruSeq (Illumina), SureSelect (Agilent), Bioanalyzer (Agilent) and TaqMan (ThermoFisher).

In some embodiments, levels of the colorectal cancer biomarker genes can be analyzed on a gene array. Microarray analysis can be performed on a customized gene array include probes corresponding to two or more of the colorectal cancer biomarkers listed in Table 1. Alternatively or in addition, microarray analysis can be carried out using commercially-available systems according to the manufacturer's instructions and protocols. Exemplary commercial systems include Affymetrix GENECHIP® technology (Affymetrix, Santa Clara, Calif.), Agilent microarray technology, and the NCOUNTER® Analysis System (NanoString® Technologies) and the BeadArray Microarray Technology (Illumina) Nucleic acids extracted from a patient's stool sample can be hybridized to the probes on the gene array. Probe-target hybridization can be detected by chemiluminescence to determine the relative abundance of particular sequences.

Levels of the colorectal cancer biomarker genes can also be analyzed by DNA sequencing. DNA sequencing can be performed by sequencing methods such as targeted sequencing, whole genome sequencing or exome sequencing. Sequencing methods can include: Sanger sequencing or high-throughput sequencing. High throughput sequencing can involve sequencing-by-synthesis, pyrosequencing, sequencing-by-ligation, real-time sequencing, nanopore sequencing, and Sanger sequencing.

In some embodiments, the extracted mRNA can be prepared for Next-generation DNA sequencing analysis. The total RNA can be extracted using QIAGEN RNeasy® Kit. The sequencing library can be generated using the Illumina® TruSeq® RNA Sample Preparation Kit v3 by following the manufacturer's protocol: briefly, polyA-containing mRNA can be first purified and fragmented from the total RNA. The first-strand cDNAs synthesis can be performed using random hexamer primers and reverse transcriptase and followed by the second strand cDNA synthesis. After the end-repair process of converting the overhangs into blunt ends of cDNAs, multiple indexing adapters can be added to the end of the double stranded cDNA and PCR performed to enrich the targets using the primer pairs specific for the gene panel and optionally the control genes. Finally the indexed libraries can be validated, normalized and pooled for sequencing on the Next-generation DNA sequencer. The Next-generation DNA sequencer can be those described herein.

Sequence-by-synthesis (SBS) can be performed using sequencing primers complementary to the sequencing element on the nucleic acid tags. The method involves detecting the identity of each nucleotide immediately after (substantially real-time) or upon (real-time) the incorporation of a labeled nucleotide or nucleotide analog into a growing strand of a complementary nucleic acid sequence in a polymerase reaction. After the successful incorporation of a label nucleotide, a signal is measured. Examples of sequence-by-synthesis methods are described in U.S. Application Publication Nos. 2003/0044781, 2006/0024711, 2006/0024678 and 2005/0100932, herein incorporated by reference. Examples of labels that can be used to label nucleotide or nucleotide analogs for sequencing-by-synthesis include, but are not limited to, chromophores, fluorescent moieties, enzymes, antigens, heavy metal, magnetic probes, dyes, phosphorescent groups, radioactive materials, chemiluminescent moieties, scattering or fluorescent nanoparticles, Raman signal generating moieties, and electrochemical detection moieties. In some embodiments, the nucleotides can be reversible terminators for example, a cleavable or photobleachable dye label as described, for example, in U.S. Pat. No. 7,427,67, U.S. Pat. No. 7,414,1163 and U.S. Pat. No. 7,057,026, the disclosures of which are incorporated herein by reference. Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199 and PCT Publication No. WO 07/010251, the disclosures of which are incorporated herein by reference in their entireties.

Pyrosequencing involves detecting the release of inorganic pyrophosphate (PPi) as particular nucleotides are incorporated into the growing strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. Each base incorporation is accompanied by release of pyrophosphate, converted to ATP by sulfurylase, which drives synthesis of oxyluciferin and the release of visible light. Because pyrophosphate release is equimolar with the number of incorporated bases, the intensity of the emitted light is proportional to the number of nucleotides added in any one step. The process can be repeated until the entire sequence is determined.

Sequencing by ligation involves a four-color sequencing by ligation process. An anchor primer is hybridized to one of four positions. Subsequently the anchor primer is enzymatically ligated to a population of degenerate nonamers that are labeled with fluorescent dyes. At any given cycle, the population of nonamers that is used is structured such that the identity of one of its positions is correlated with the identity of the fluorophore attached to that nonamer. Exemplary systems and methods which can be utilized with the methods and systems described herein are described in U.S. Pat. Nos. 6,969,488, 6,172,218, and 6,306,597, the disclosures of which are incorporated herein by reference in their entireties.

Real-time sequencing involves sequencing a target nucleic acid molecule by the temporal addition of bases via a polymerization reaction that is measured on a molecule of a nucleic acid, i.e., the activity of a nucleic acid polymerizing enzyme on the template nucleic acid molecule to be sequenced is followed in real time. The sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is then identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In one embodiment, Sanger sequencing can be performed on a MegaBACE™ capillary electrophoresis instrument (Molecular Dynamics/GE Healthcare) per the manufacturer's instructions. In one aspect, Sanger sequencing can be performed on an ABI 3730x1 instrument, or 3700 Genetic Analyzer (Applied Biosystems/Life Technology/Thermo Fisher) per the manufacturer's instructions. In one embodiment, Sanger sequencing can be performed on an IntegenX RapidHit™ system (IntegenX). In one embodiment, Sanger sequencing can be performed on a polyacrylamide slab gel using electrophoresis using gels and analytical instrumentation.

In one embodiment, high-throughput sequencing can be performed using commercially available products employing a sequencing-by-synthesis strategy. Such products include those sold by Illumina, Inc. (San Diego, Calif.). Such products include the Genome Analyzer™, GA II™, HiSeq 2000™, HiSeq 2500™, HiSeq 3000™, HiSeq 4000™, the MiSeq™, MiSeqDX™, NextSeq™, NextSeq 500™, HiSeq X Ten™, HiSeq X Five™, MiniSeq, and all future developments therefrom.

In one embodiment, high-throughput sequencing can be performed using commercially available products from Life Technologies/Thermo Fisher (San Diego, Calif.) per the manufacturer's instructions. Such products include the Ion Torrent PGM™, Ion Torrent Proton™, and the Solid Sequencer™.

In one embodiment, Next-generation high-throughput sequencing can be performed using commercially available products from Pacific Biosciences (Menlo Park, Calif.) per the manufacturer's instructions. Such products include the RS II™.

In one embodiment, Next-generation high-throughput sequencing can be performed using the systems offered by Complete Genomics, Inc. Libraries of target nucleic acids can be prepared where target nucleic acid sequences are interspersed approximately every 20 by with adaptor sequences. The target nucleic acids can be amplified using rolling circle replication to generate ‘DNA nanoballs,’ and the amplified target nucleic acids can be used to prepare an array of target nucleic acids. Methods of sequencing such arrays include sequencing by ligation, in particular, sequencing by combinatorial probe-anchor ligation (cPAL). In some embodiments using the cPAL method, about 10 contiguous bases adjacent to an adaptor may be determined. A pool of probes comprising four discrete labels for each base (A, C, T, G) is used to read the positions adjacent to each adaptor. A separate pool is used to read each position. A pool of probes and an anchor specific to a particular adaptor can be delivered to the target nucleic acid in the presence of a ligase. The anchor sequence hybridizes to the adaptor, and a probe hybridizes to the target nucleic acid adjacent to the adaptor. The anchor sequence and probe are ligated to one another. The hybridization is detected and the anchor-probe complex is removed. A different anchor and pool of probes is delivered to the target nucleic acid in the presence of the ligase.

The sequencing methods described herein can be carried out in multiplex formats such that multiple different target nucleic acids are manipulated simultaneously. In some embodiments, different target nucleic acids can be treated in a common reaction vessel or on a surface of a particular substrate, enabling convenient delivery of sequencing reagents, removal of unreacted reagents and detection of incorporation events in a multiplex manner. In some embodiments where surface-bound target nucleic acids are involved, the target nucleic acids may be in an array format. In an array format, the target nucleic acids may be typically coupled to a surface in a spatially distinguishable manner. For example, the target nucleic acids may be bound by direct covalent attachment, attachment to a bead or other particle or associated with a polymerase or other molecule that is attached to the surface. The array may include a single copy of a target nucleic acid at each site (also referred to as a feature) or multiple copies having the same sequence can be present at each site or feature. Multiple copies are produced by amplification methods such as, bridge amplification or emulsion PCR.

In some embodiments, a normalization step can be used to control for nucleic acid recovery and variability between samples. In some embodiments, a defined amount of exogenous control nucleic acids can be added (“spiked in”) to the extracted human nucleic acids. The exogenous control nucleic acid can be a nucleic acid having a sequence corresponding to one or more human sequences. Alternatively or in addition, the exogenous control nucleic acid can have a sequence corresponding to the sequence found in another species, for example a bacterial sequence such as a Bacillus subtilis sequence. In some embodiments, the methods can include determining the levels of one or more housekeeping genes. In some embodiments, the methods can include normalizing the expression levels of the biomarkers in Table 1 to the levels of the housekeeping genes.

The methods include the step of determining whether the measured expression levels of two or more colorectal cancer biomarker genes selected from the panels in Table 1 are different from the measured expression levels of the two or more colorectal cancer biomarker genes in a control sample. A difference in expression level can be an increase or a decrease. We may use the terms “increased”, “increase” or “up-regulated” to generally mean an increase in the level of a colorectal cancer biomarker by a statistically significant amount. In some embodiments, an increase can be an increase of at least 10% as compared to a control sample or reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a reference level.

We may use the terms “decrease”, “decreased”, “reduced”, “reduction” or “down-regulated” to refer to a decrease in the level of a colorectal cancer biomarker by a statistically significant amount. In some embodiments, a decrease can be a decrease of at least 10% as compared to a reference level, for example a decrease of at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a reference level.

The statistical significance of an increase in a colorectal cancer biomarker or a decrease in a colorectal cancer biomarker can be expressed as a p-value. Depending upon the specific colorectal cancer biomarker p-value can be less than 0.01, less than 0.005, less than 0.002, less than 0.001, or less than 0.0005.

A control sample can be a reference sample. The reference sample can be a sample obtained from the subject at one or more previous points in time. Alternatively or in addition, a reference sample can be a standard reference level of particular colorectal cancer biomarkers derived from a larger population of individuals. The reference population may include individuals of similar age, body size, ethnic background or general health as the subject. Thus, the levels of colorectal cancer biomarkers can be compared to values derived from healthy individuals, i.e. individuals who are not suffering from colorectal cancer or who are not at risk for colorectal cancer. Healthy individuals can include, for example, individuals who have tested negative in a fecal occult blood test (FOBT), a fecal immunochemical test (FIT), a DNA test or a colonoscopy within the last five years. A reference sample can also be a sample obtained from a population of individuals who are in remission. The population of individuals in remission can include individuals having a similar kind or stage of colorectal cancer and who have received similar therapeutic treatment.

The level of two or more colorectal cancer biomarker genes selected from Table 1 can be analyzed in a subject at risk for or having colorectal cancer. All of the 564 colorectal cancer biomarker genes listed in Table 1 form a panel (“Panel A”). A subset of 277 colorectal cancer biomarker genes in Table 1 comprise Panel B. A subset of 95 colorectal cancer biomarker genes in Table 1 comprise Panel C. A subset of 39 colorectal cancer biomarker genes in Table 1 comprise Panel D. A subset of 22 colorectal cancer biomarker genes in Table 1 comprise Panel E. In some embodiments, the two or more biomarkers can include combinations of 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 120, 140, 160, 180, 200, 225, 250, 275, 300, 325, 350, 375, 400, 425, 450, 475, 500, 525, 550, 575 or more of the markers in Table 1. In some embodiments, the two or more colorectal cancer biomarkers can include combinations of 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 120, 140, 160, 180, 200, 220, 240, 260, 270, 280, 285 or more of the colorectal cancer markers in Panel B. In some embodiments, the two or more colorectal cancer biomarkers can include combinations of 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, or more of the markers in Panel C. In some embodiments, the two or more colorectal cancer biomarkers can include 2, 3, 4, 5, 6, 7, 8, 9, 10, 11, 12, 13, 14, 15, 16, 17, 18, 19, 20, 21, 22, 23, 24, 25, 30, 35, or more of the colorectal cancer markers in Panel D. In some embodiments, the two or more colorectal cancer biomarkers can include 2, 3, 4, 5, 6, 7, 8, 9, 10, 11, 12, 13, 14, 15, 16, 17, 18, 19, 20, 21, or more of the colorectal cancer markers in Panel E. In some embodiments the two or more colorectal cancer biomarkers can include a panel of markers selected from the colorectal cancer biomarkers having the mRNA Accession or Ensembl Numbers AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, and uc021uck.1. In some embodiments, the two or more colorectal cancer biomarkers can include a panel of markers selected from the colorectal cancer biomarkers having the mRNA Accession or Ensembl Numbers AK024621, NR_002589, TCONS_l2_00011049-XLOC_l2_005952, AK022857, NR_030630, NM_002165, ENST00000459148, NR_001281, OTTHUMT00000051727, ENST00000365621, BC039358, NM_030876, ENST00000390298, TCONS_00014878-XLOC_006946, TCONS_00028807-XLOC_013883, linc_luo_1487, TCONS_l2_00017903-XLOC_l2_009470, TCONS_00009728-XLOC_004927, ENST00000408390, ENST00000384552, uc021uck.1, TCONS_00017621-XLOC_008311, ENST00000364506, NM_032551, ENST00000554665, AF086063, ENST00000528885, NR_039685, ENST00000557910, AK090788, NR_033379, NR_033379, NR_033379, NR_033379, NR_033379, ENST00000384633, OTTHUMT00000052823, BC008667, NM_207410, X64978, TCONS_00028080-XLOC_013828, ENST00000516724.

Algorithms for determining diagnosis, status, or response to treatment, for example, can be determined for particular clinical conditions. The algorithms used in the methods provided herein can be mathematic functions incorporating multiple parameters that can be quantified using, without limitation, medical devices, clinical evaluation scores, or biological/chemical/physical tests of biological samples. Each mathematic function can be a weight-adjusted expression of the levels (e.g., measured levels) of parameters determined to be relevant to a selected clinical condition. Because of the techniques involved in weighting and assessing multiple marker panels, computers with reasonable computational power can be used to analyze the data.

Thus, the method of diagnosis can include obtaining a stool sample from a patient at risk for or suspected of having colorectal cancer; determining the expression of two or more colorectal cancer biomarker genes selected from Table 1 and providing a test value by the machine learning algorithms that incorporate a plurality of colorectal cancer biomarker genes selected from any of the panels of colorectal cancer biomarker genes with a predefined coefficient. A significant change in expression of a plurality of colorectal cancer biomarker genes relative to the value of reference sample, for example, a population of healthy individuals, indicates an increased likelihood that the patient has colorectal cancer. In some embodiments, the expression levels measured in a sample are used to derive or calculate a probability or a confidence score. This value may be derived from expression levels. Alternatively or in addition, the value can be derived from a combination of the expression value with other factors, for example, the patient's medical history, age, and genetic background. In some embodiments, the method can further comprise the step of communicating the test value to the patient.

Standard computing devices and systems can be used and implemented, e.g., suitably programmed, to perform the methods described herein, e.g., to perform the calculations needed to determine the values described herein. Computing devices include various forms of digital computers, such as laptops, desktops, mobile devices, workstations, personal digital assistants, servers, blade servers, mainframes, and other appropriate computers. In some embodiments, the computing device is a mobile device, such as personal digital assistant, cellular telephone, smartphone, tablet, or other similar computing device.

In some embodiments, a computer can be used to communicate information, for example, to a healthcare professional. Information can be communicated to a professional by making that information electronically available (e.g., in a secure manner). For example, information can be placed on a computer database such that a health-care professional can access the information. In addition, information can be communicated to a hospital, clinic, or research facility serving as an agent for the professional. Information transferred over open networks (e.g., the internet or e-mail) can be encrypted. Patient's gene expression data and analysis can be stored in the cloud with encryption. The method 256-bit AES with tamper protection can be used for disk encryption; SSL protocol preferably can ensure protection in data transit, and key management technique SHA2-HMAC can allow authenticated access to the data. Other secure data storage means can also be used.

The results of such analysis above can be the basis of follow-up and treatment by the attending clinician. If the expression level of two or more colorectal cancer biomarker genes selected from Table 1 is not significantly different from the expression level of the same two or more colorectal cancer biomarkers in a control sample, for example, a reference sample, the clinician may determine that the patient is presently not at risk for colorectal cancer. Such patients can be encouraged to return in the future for rescreening. The methods disclosed herein can be used to monitor any changes in the levels of the colorectal cancer markers over time. A subject can be monitored for any length of time following the initial screening and/or diagnosis. For example, a subject can be monitored for at least 2, 4, 6, 8, 10, 12, 14, 16, 18, 20, 25, 30, 35, 40, 45, 50, 55, or 60 months or more or for at least 1, 2, 3, 4, 5, 6, 7, 8, 9, 10, 11, 12, 13, 14, 15, 16, 17, 18, 19, 20 or more years.

The methods and compositions disclosed herein are useful for selecting a clinical plan for a subject at risk for or suffering from colorectal cancer. The clinical plan can include administration of further diagnostic procedures, for example, a fecal occult blood test, a fecal immunochemical test, or a colonoscopy to remove polyps. In some embodiments, the clinical plan can include a method of treatment. In some embodiments, the methods include methods of selecting a treatment for a subject having colorectal cancer. If the expression level of two or more colorectal cancer biomarker genes selected from Table 1 is significantly different from the expression level of the same two or more colorectal cancer biomarker genes in a control sample, for example, a reference sample, the patient may have colorectal cancer. In these instances, further screening may be recommended, for example, increased frequency of screening using the methods disclosed herein, as well as a fetal occult blood test, a fecal immunochemical test, and/or a colonoscopy. In some embodiments, treatment may be recommended, including, for example, a colonoscopy with removal of polyps, chemotherapy, or surgery, such as bowel resection. Thus, the methods can be used to determine the level of expression of two or more colorectal cancer biomarker genes and then to determine a course of treatment. A subject, that is a patient, is effectively treated whenever a clinically beneficial result ensues. This may mean, for example, a complete resolution of the symptoms of a disease, a decrease in the severity of the symptoms of the disease, or a slowing of the disease's progression. These methods can further include the steps of a) identifying a subject (e.g., a patient and, more specifically, a human patient) who has colorectal cancer; and b) providing to the subject an anticancer treatment, for example, a therapeutic agent, surgery, or radiation therapy. An amount of a therapeutic agent provided to the subject that results in a complete resolution of the symptoms of a disease, a decrease in the severity of the symptoms of the disease, or a slowing of the disease's progression is considered a therapeutically effective amount. The present methods may also include a monitoring step to help optimize dosing and scheduling as well as predict outcome. Monitoring can also be used to detect the onset of drug resistance, to rapidly distinguish responsive patients from nonresponsive patients or to assess recurrence of a cancer. Where there are signs of resistance or nonresponsiveness, a clinician can choose an alternative or adjunctive agent before the tumor develops additional escape mechanisms.

The methods disclosed herein can also be used in combination with conventional methods for diagnosis and treatment of colorectal cancer. Thus, the diagnostic methods can be used along with standard diagnostic methods for colorectal cancer. For example, the methods can be used in combination with a fecal occult blood test, a fecal immunochemical test, or a colonoscopy. The methods can also be used with other colorectal cancer markers, for example, KRAS, NRAS, BRAF, CEA, CA 19-9, p53, MSL, DCC and MMR.

The diagnostic methods disclosed herein can also be used in combination with colorectal cancer treatments. Colorectal cancer treatment methods fall into several general categories: surgery, chemotherapy, radiation therapy, targeted therapy and immunotherapy. Surgery can include colectomy, colostomy along with partial hepatectomy, or protectomy. Chemotherapy can be systemic chemotherapy or regional chemotherapy in which the chemotherapeutic agents are placed in direct proximity to an affected organ. Exemplary chemotherapeutic agents can include 5-fluorouracil, oxaliplatin or derivatives thereof, irinotecan or a derivative thereof, leucovorin, or capecitabine, mitomycin C, cisplatin and doxorubicin. Radiation therapy can be external radiation therapy, using a machine to direct radiation toward the cancer or internal radiation therapy in which a radioactive substance is placed directly into or near the colorectal cancer. Targeted agents can include anti-angiogenic agents such as bevacizumab) or EGFR inhibitor monoclonal antibody (cetuximab, panitumumab), ramuciramab (anti-VEGFR2), aflibercept, regorafenib, tripfluridine-tipiracil or a combination thereof. Targeted agents can also be combined with standard chemotherapeutic agents. Immunotherapy can include administration of specific antibodies, for example anti-PD-1 antibodies, anti-PD-L-1 antibodies, and time-CTLA-4 antibodies, anti-CD 27 antibodies; cancer vaccines, adoptive cell therapy, oncolytic virus therapies, adjuvant immunotherapies, and cytokine-based therapies. Other treatment methods include stem cell transplantation, hyperthermia, photodynamic therapy, blood product donation and transfusion, or laser treatment.

### Articles of Manufacture

Also provided are kits for detecting and quantifying selected colorectal cancer biomarkers in a biological sample, for example, a stool sample. Accordingly, packaged products (e.g., sterile containers containing one or more of the compositions described herein and packaged for storage, shipment, or sale at concentrated or ready-to-use concentrations) and kits, are also within the scope of the invention. A product can include a container (e.g., a vial, jar, bottle, bag, microplate, microchip, or beads) containing one or more compositions of the invention. In addition, an article of manufacture further may include, for example, packaging materials, instructions for use, syringes, delivery devices, buffers or other control reagents.

The kit can include a compound or agent capable of detecting RNA corresponding to two or more of the colorectal cancer biomarker genes selected from Table 1 in a biological sample; and a standard; and optionally one or more reagents necessary for performing detection, quantification, or amplification. The compounds, agents, and/or reagents can be packaged in a suitable container. The kit can further comprise instructions for using the kit to detect and quantify nucleic acid. For example, the kit can include: (1) a probe, e.g., an oligonucleotide, e.g., a detectably labeled oligonucleotide, which hybridizes to a nucleic acid sequence corresponding to a two or more of the colorectal biomarker genes selected from Table 1 or (2) a pair of primers useful for amplifying a nucleic acid molecule corresponding to two or more of the colorectal biomarker genes selected from Table 1. The kit can further include probes and primers useful for amplifying one or more housekeeping genes. The kit can also include a buffering agent, a preservative, and/or a nucleic acid or protein stabilizing agent. The kit can also include components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit. In some embodiments the kits can include primers or oligonucleotide probes specific for one or more control markers. In some embodiments, the kits include reagents specific for the quantification of two or more of the colorectal biomarkers selected from Table 1.

In some embodiments, the kit can include reagents specific for the separation of human cells from bacterial cells and other stool components and extraction of human mRNA from a patient's stool sample. Thus the kit can include buffers, emulsion beads, silica beads, stabilization reagents and various filters and containers for centrifugation. The kit can also include instructions for stool handling to minimize contamination of samples and to ensure stability of human mRNA in the stool sample. The kit can also include items to ensure sample preservation, for example, coolants or heat packs. In some embodiments, the kit can include a stool collection device.

The product may also include a legend (e.g., a printed label or insert or other medium describing the product's use (e.g., an audio- or videotape or computer readable medium)). The legend can be associated with the container (e.g., affixed to the container) and can describe the manner in which the reagents can be used. The reagents can be ready for use (e.g., present in appropriate units), and may include one or more additional adjuvants, carriers or other diluents. Alternatively, the reagents can be provided in a concentrated form with a diluent and instructions for dilution.

### EXAMPLES

### Example 1: Materials and Methods

Stool Collection:

Patients were asked to defecate into a bucket that fit over a toilet seat and store in the freezer until they were transported to the Kharkiv National Medical University in the Ukraine. The stool was aliquotted into 50 mL conical tubes and stored at −80° C. The samples were shipped from the university on dry ice to Capital Biosciences (Gaithersburg, Md.) and immediately transferred to a −80° C. freezer. From there, the samples were shipped on dry ice to Washington University School of Medicine where they were stored in a −80° C. freezer until extraction.

RNA Extraction.

Each sample was placed into a conical tube with approximately 10 zirconium/silica beads. Approximately 1,000 mg of stool were added to each tube. An additional 3 mL of Hanks Balanced Salt Solution (HBSS) (Sigma-Aldrich) were added to each tube and the solution was vortexed at low speed for 10 minutes. The solution volume was increased to 10 mL and incubated at 4° C. for 10 minutes with rotation. The solution was centrifuged at 1000 rpm at 4° C. for 10 minutes and the supernatant was removed. This procedure was repeated and the supernatant removed. Approximately 2 mL of EasyMag® Lysis Buffer (bioMerieux) was added to the pellet and the solution was centrifuged at 3500 rpm at 20° C. for 10 minutes. The solution was transferred to EasyMag® Disposable cartridges (bioMerieux) and 75 uL of EasyMag® Magnetic Silica (bioMerieux) was added. The beads were mixed into the solution for 1 minute. Then the total nucleic acid was separated out and eluted into a 110 uL solution. Nucleic acids were quantified by UV/vis spectroscopy.

### Example 2: Human mRNA Levels in Stool Samples

Stool samples were obtained from 10 patients with colorectal cancer and 10 control patients. Healthy controls were patients with no history of colorectal cancer, irritable bowel disease, celiac disease, irritable bowel syndrome, diarrhea within the last 20 days or any other gastrointestinal disease. Colorectal cancer donors consisted of patients who had been diagnosed with Stage IV colorectal cancer via biopsy within the last month and had not yet received any post-biopsy treatment, which includes chemotherapy, radiation, or surgery. The healthy controls were matched with cancer patients based on gender and age brackets (50-60 years, 60-70 years, 70-80 years and 80-90 years). The patients used for this study were consented by Capital Biosciences (Gaithersburg, Md.). All stool samples were collected and frozen at −80° C. within 24 hours of defecation. The samples were stored at −80° C. until they were shipped to the Washington University School of Medicine for extraction and analysis. The Washington University School of Medicine Internal Review Board provided ethical oversight for this study.

Human mRNA levels in stool samples were measured as follows. Samples were treated with DNase at 37° C. for 30 minutes. A 500 μL aliquot of lysis buffer was added and the sample was transferred to a new cartridge. An additional 1.5 mL of lysis buffer was added to the cartridge along with 40 μL of EasyMag® Magnetic Silica. Samples were loaded into 50 μL and stored overnight at 4° C.

GADPH levels were assayed by reverse transcription-polymerase chain reaction (RT-PCR) using Droplet Digital™ PCR (ddPCR™) Technology. A master mix/probe solution formulated according to Table 2. In 1.2 ml of the MasterMix, there were 0.075 units per μl Taq DNA polymerase, reaction buffer, 4 mM MgCl2, 0.4 mM of each dNTP (dATP, dCTP, dGTP, dTTP) (Bio Rad). The GAPDH PrimePCR™ FAM Probe (Bio Rad) was used for the primer annealing.

TABLE 2
RT-PCR Master Mix

| | Volume | |
|---|---|---|
| Reagent | per well | Total |
| RNA | 2μ | |
| MasterMix | 25.6μ | 345.6μ |
| (BioRad) | | |
| Probe | 2.5μ | 67.5μ |
| Water | 7.7μ | 207.9μ |

A 20 μL aliquot of the RNA mix was added to the middle well on the cartridge followed by 70 μL of Oil Droplet solution (BioRad), and the samples run on the Droplet generator instrument (BioRad). A 40 μL aliquot of solution was transferred to a PCR plate and the plate was transferred to a thermocycler. After completion of the PCR reaction the values for each sample were determined in a ddPCR reader (BioRad).

The results of these analyses are shown in Tables 3 and 4. As shown in Tables 3 and 4, GADPH mRNA levels in stool samples from cancer patients were generally higher than those from control patients. Overall, the data shown in Tables 3 and 4 reflect the increased levels of human colorectal cancer cells in stool from colorectal cancer patients.

TABLE 3
GADPH mRNA Levels in Stool Samples from Cancer Patients
Cancer Samples

| Sample number | GADPH/ug |
|---|---|
| 1 | 0.3422131 |
| 2 | 74.0234375 |
| 3 | 1.5642077 |
| 4 | 7.5236967 |
| 5 | 64.4067797 |
| 6 | 46.8750000 |
| 7 | 12.1284965 |
| 8 | 1.2500000 |
| 9 | 0.3959732 |
| 10 | 0.5090909 |
| 5 (duplicate) | 70.6043956 |
| 9 (duplicate) | 0.5241117 |
| Average | 24.3456169 |

TABLE 4
GDAPH mRNA Levels in Stool Samples from Control Patients
Control Samples

| Sample number | GADPH/ug |
|---|---|
| 1N | 0.6885027 |
| 2N | 0.3251295 |
| 3N | 1.8846154 |
| 4N | 24.8684211 |
| 5N | 0.6842105 |
| 6N | 2.4141221 |
| 7N | 1.1064593 |
| 8N | 2.514045 |
| 9N | 1.0451977 |
| 10N | |
| 8N (duplicate) | 2.3573826 |
| 2N (duplicate) | 3.2542194 |
| Average | 3.4285387 |

### Example 3: MicroArray Analysis

The samples were sent to the Genome Technology Access Center (GTAC) and further analyzed for RNA content and RNA quality. To assess the RNA quality, the RNA Integrity Number (RIN) values were determined. The RIN values ranged from 1.00-4.50. Samples were only selected with a RIN score of greater than 1.70. The quantity of RNA was assessed by evaluating the RNA banding on gel electrophoresis. Samples were selected if the band was visible by the naked eye. As a result, fifteen samples were selected in total; eight from the colorectal cancer cohort and seven samples were selected from the healthy control to run on MicroArray. RNA samples were analyzed by MicroArray analysis using a MicroArray chip obtained from Affymetrix. The MicroArray chip contained probes corresponding to 42,000 different human sequences.

The RNA samples were analyzed by MicroArray analysis using a GeneChip® Human Transcriptome Array 2.0 (Affymetrix). The analysis was performed using the GeneChip® Human Transcriptome Pico Assay 2.0 (Affymetrix) according to the supplier's directions. These chips were read using a GeneChip® Scanner 3000 7G (Affymetrix). The raw data were in a CEL format that stores luminance intensities of the probesets and associated intensity calculation, such as standard deviation of intensity, pixel count and outlier flag. The CEL files were consolidated and analyzed.

The raw CEL files are processed and the expression levels on the probe sets were normalized and log 2 transformed using the RMA (Robust Multi-array Average) method. Fifteen output samples were obtained. We used the Pos vs Neg AUC value, which compares the detection of positive controls against the false detection of negative controls, as the overall data quality measurement. Samples with the value below 0.79 were removed. We used the RLE (relative log expression) values to access the biological variance across arrays, as the expressions on most probesets were assumed to be unchanged. Samples with RLE values greater than 0.23 were removed. The control probesets were then removed. Twelve output samples were valid for downstream analysis.

Differential expression analysis was performed using LIMMA (Linear Models for MicroArray Data) differential expression analysis. We used the R Limma library to estimate the significantly differentially expressed (DE) genes. We first created an appropriate contrast matrix for cancer-normal comparison from the corresponding known sample labels. Then we fit a linear model for each gene according to the 12 valid arrays and estimates coefficients and standard errors of the model. We computed the empirical Bayes smoothness method to shrink high or low variability genes towards the average level among all genes. We then computed moderated t-statistics and log-odds ratios. Genes with p-value lower than specific threshold were reported.

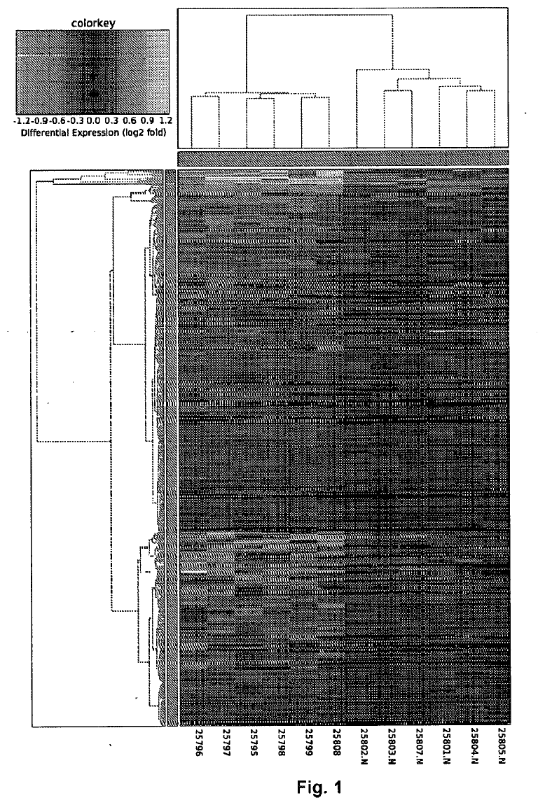

The results of this analysis are shown in FIGS. 1-6 and in Table 1. We observed a statistically significant difference in the levels of certain mRNAs in stool samples from colorectal cancer patients compared to stool samples from control patients. Table 1 lists the 564 colorectal cancer biomarkers identified by this analysis. The measured expression levels of the colorectal cancer biomarkers listed in Table 1 were statistically significantly different in stool samples from colorectal cancer patients as compared to stool samples from control patients based on p-values from a moderated t-test. The p-values of the colorectal cancer biomarkers shown in Table 1 ranged in statistical significance from 0.0005 to 0.01. A heat map of the 564 colorectal cancer biomarkers shown in Table 1 is presented in FIG. 1.

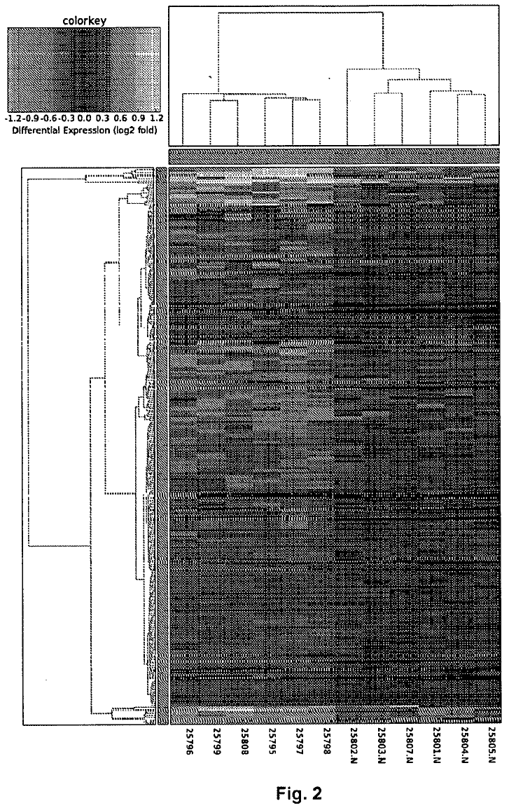

A subset of 277 colorectal cancer biomarker genes in Table 1 comprise Panel B The colorectal cancer biomarker genes in Panel B showed measured expression levels that were statistically significantly different from the measured expression levels of the same colorectal cancer biomarkers in control samples at a p value of 0.005. A heat map of the 277 colorectal cancer biomarkers in Panel B is presented in FIG. 2.

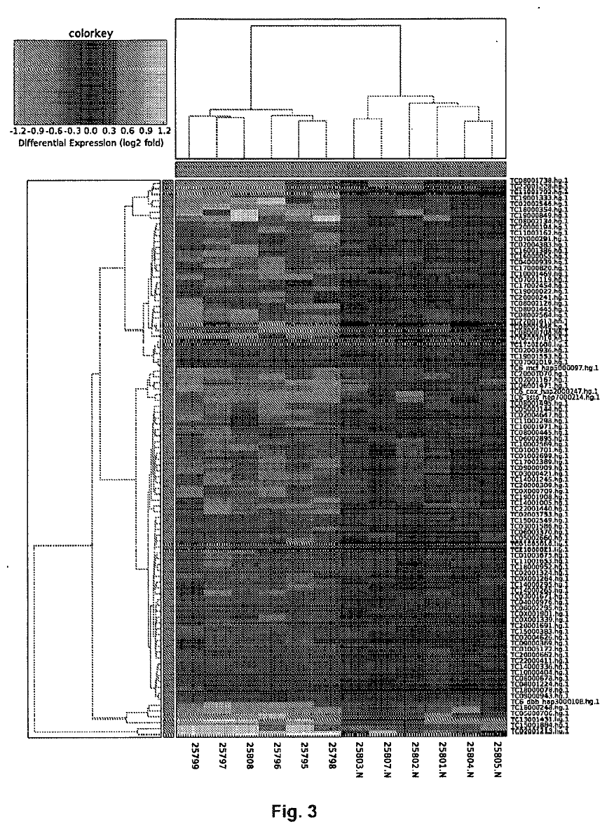

A subset of 95 colorectal cancer biomarker genes in Table 1 comprise Panel C. The colorectal cancer biomarker genes in Panel C showed measured expression levels that were statistically significantly different from the measured expression levels of the same colorectal cancer biomarkers in control samples at a p value of 0.002. A heat map of the 95 colorectal cancer biomarkers in Panel C is presented in FIG. 3.

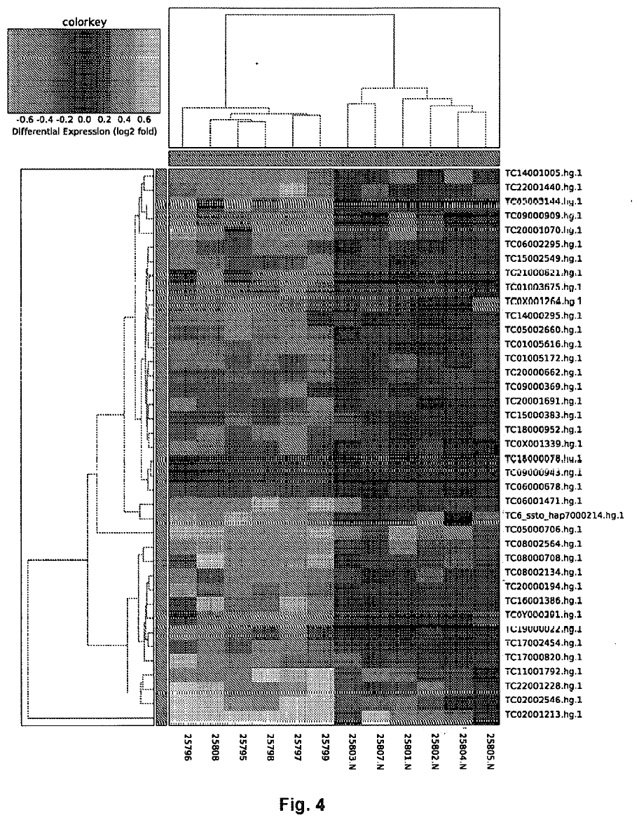

A subset of 39 colorectal cancer biomarker genes in Table 1 comprise Panel D. The colorectal cancer biomarker genes in Panel D showed measured expression levels that were statistically significantly different from the measured expression levels of the same colorectal cancer biomarkers in control samples at a p value of 0.001. A heat map of the 39 colorectal cancer biomarkers in Panel D is presented in FIG. 4.

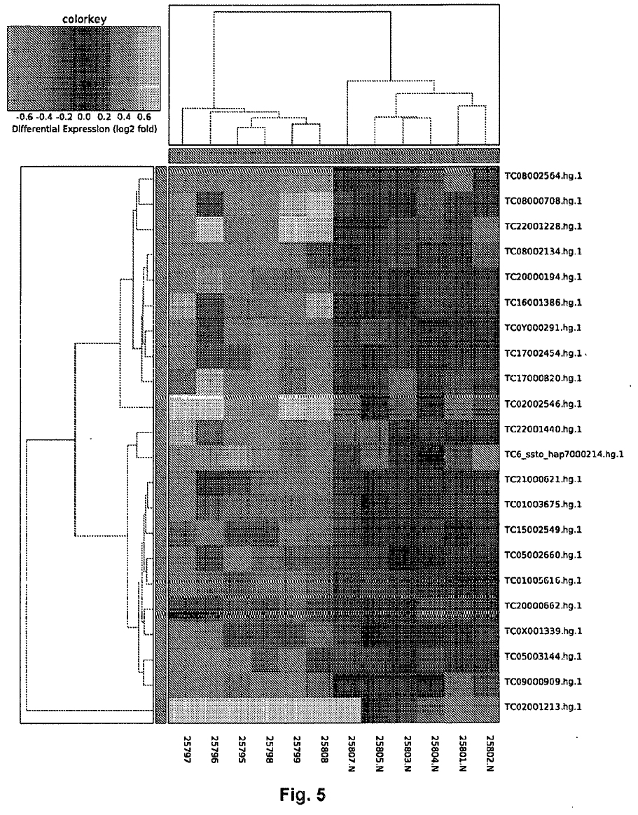

A subset of 22 colorectal cancer biomarker genes in Table 1 comprise Panel E. The colorectal cancer biomarker genes in Panel E showed measured expression levels that were statistically significantly different from the measured expression levels of the same colorectal cancer biomarkers in control samples at a p value of 0.0005. A heat map of the 22 colorectal cancer biomarkers in Panel E is presented in FIG. 5.

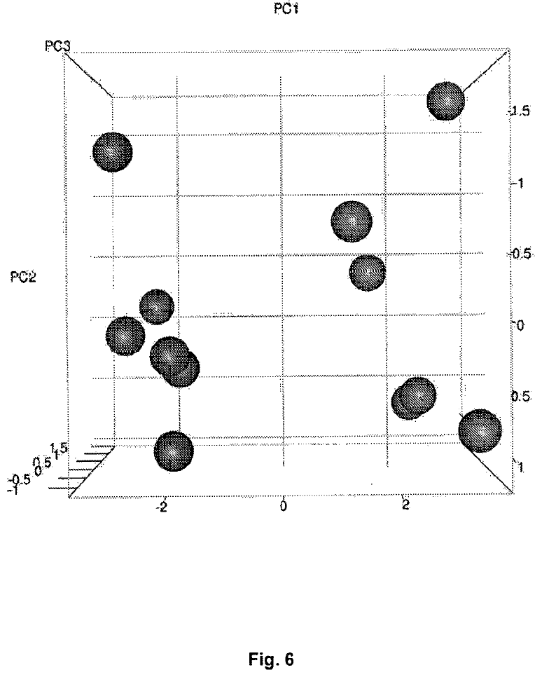

A principal component analysis of the 564 colorectal cancer biomarkers identified by this method is shown in FIG. 6. This analysis consolidates all variables in the principal component analysis and clusters populations into a three-dimensional plot. Cancer samples, highlighted in green, all clustered into a distinct location in space based on similarities between expression levels. Conversely, normal controls, highlighted in red, had a wider spread of clustering detailing the variation than can be seen with the general population. Overall, however, these two populations were specially distinct, representing the ability of the colorectal cancer biomarker genes to effectively segregate the two populations.

1. A method of detecting colorectal cancer in a subject, the method comprising:
a) measuring the level of expression of two or more colorectal cancer biomarker genes selected from any of the colorectal cancer biomarker genes listed in Table 1 (Panel A) in a biological sample from the subject;
b) comparing the measured expression level of the two or more colorectal cancer biomarker genes in the sample with the measured expression level of the two or more colorectal cancer biomarker genes in a control sample, wherein a difference in the measured expression level of the two more genes in the biological sample relative to the measured expression level of the two or more genes in the control sample indicates that the subject has colorectal cancer.

2. The method of claim 1, wherein the two or more colorectal cancer biomarker genes are selected from the colorectal cancer biomarker genes listed in Panel B, Panel C, Panel D, or Panel E.

3-7. (canceled)

8. The method of claim 1, wherein the biological sample is a stool sample.

9. The method of claim 1, wherein the expression level comprises expression of an RNA selected from the group consisting of total RNA, mRNA, ncRNA, rRNA, smRNA, and snoRNA.

10. The method of claim 1, wherein the measuring step comprises microarray analysis, reverse transcription polymerase chain reaction (RT-PCR), or nucleic acid sequencing.

11-13. (canceled)

14. A method of determining whether a subject is at risk for colorectal cancer, the method comprising:
a) measuring the level of expression of two or more colorectal cancer biomarker genes selected from any of the colorectal cancer biomarker genes listed in Table 1 (Panel A) in a biological sample from the subject;
b) comparing the measured expression level of the two or more colorectal cancer biomarker genes in the sample with the measured expression level of the two or more colorectal cancer biomarker genes in a control sample, wherein a difference in the measured expression level of the two or more genes in the biological sample relative to the measured expression level of the two or more genes in the control sample indicates that the subject is at risk for colorectal cancer.

15. The method of claim 14, wherein the two or more colorectal cancer biomarker genes are selected from the colorectal cancer biomarker genes listed in Panel B, Panel C, Panel D, or Panel E.

16-20. (canceled)

21. The method of claim 14, wherein the biological sample is a stool sample.

22. The method of claim 14, wherein the expression level comprises expression of an RNA selected from the group consisting of total RNA, mRNA, tRNA, rRNA, ncRNA, smRNA, and sno RNA.

23. The method of claim 14, wherein the measuring step comprises microarray analysis, reverse transcription polymerase chain reaction (RT-PCR), or nucleic acid sequencing.

24-26. (canceled)

27. A method of selecting a clinical plan for a subject having or at risk for colorectal cancer, the method comprising:
a) measuring the level of expression of two or more colorectal cancer biomarker genes selected from any of the colorectal cancer biomarker genes listed in Table 1 (Panel A) in a biological sample from the subject;
b) comparing the measured expression level of the two or more colorectal cancer biomarker genes in the sample with the measured expression level of the two or more colorectal cancer biomarker genes in a control sample, wherein a difference in the measured expression level of the two or more genes relative to the measured expression level of the two or more genes in the control sample indicates that the subject has or is at risk for colorectal cancer; and
c) selecting a clinical plan based on step b.

28. The method of claim 27, wherein the two or more colorectal cancer biomarker genes are selected from the colorectal cancer biomarker genes listed in Panel B, Panel C, Panel D, or Panel E.

29-33. (canceled)

34. The method of claim 27, wherein the biological sample is a stool sample.

35. The method of claim 27, wherein the expression level comprises expression of an RNA selected from the group consisting of total RNA, mRNA, tRNA, rRNA, ncRNA, smRNA, and sno RNA.

36. The method of claim 27, wherein the measuring step comprises microarray analysis, reverse transcription polymerase chain reaction (RT-PCR), or nucleic acid sequencing.

37. (canceled)

38. The method of claim 27, wherein the clinical plan comprises a diagnostic procedure or a treatment.

39. The method of claim 38, wherein the diagnostic procedure comprises a fecal occult blood test, a fecal immunochemical test, or a colonoscopy.

40. The method of claim 38, wherein the treatment comprises surgery, chemotherapy, radiation therapy, targeted therapy, or immunotherapy.

41. The method of claim 40, wherein the chemotherapy comprises administration of 5-fluorouracil, leucovorin, capecitabine, oxaliplatin, irinotecan or a combination thereof.

42. The method of claim 40, wherein the targeted therapy comprises administration of bevacizumab (anti-VEGF), ramuciramab (anti-VEGFR2), aflibercept, regorafenib, cetuximab (anti-EGFR), panitumumab, tripfluridine-tipiracil or a combination thereof.

43-45. (canceled)
